(19) United States
(12) Patent Application Publication
(10) Pub. No.: US 2021/0162007 A1
(43) Pub. Date: Jun. 3, 2021

(54) MODULATING NUCLEAR RECEPTORS AND METHODS OF USING SAME

(71) Applicant: President and Fellows of Harvard College, Cambridge, MA (US)

(72) Inventors: Yanbo Zhang, Malden, MA (US); Christophe Benoist, Brookline, MA (US); Diane Mathis, Brookline, MA (US)

(21) Appl. No.: 17/046,447

(22) PCT Filed: Apr. 8, 2019
(86) PCT No.: PCT/US19/26329

§ 371 Date: Oct. 9, 2020

Related U.S. Application Data
(60) Provisional application No. 62/654,936, filed on Apr. 9, 2018.

(51) Int. Cl. A61K 38/17; A61K 31/201; A61K 31/203

(52) U.S. Cl. CPC A61K 38/1783; A61K 31/203; A61K 31/201

(57) ABSTRACT

The invention provides compositions and methods of modulating and regulating the activity of, expression of, or level of Nr2c2, to thereby modulate T regulatory cell (Treg) differentiation, function, activity, or maturation, or combinations of these biological activities. Such modulation may be useful in methods of treating and preventing cancer, inflammation, and inflammatory diseases, among other diseases, conditions, and illnesses.

### RELATED APPLICATIONS

This application claims priority to U.S. Provisional Application 62/654936, filed Apr. 9, 2018, which is incorporated herein by reference in its entirety.

### GOVERNMENT SUPPORT

This invention was made with government support under Grant Number AI51530, awarded by the National Institutes of Health. The government has certain rights in the invention.

### BACKGROUND OF THE INVENTION

Nuclear receptors (NR) are a large family of transcription factors with fundamental roles in the development and the specification of many tissues. First discovered as underpinning endocrine communication between organs through steroid hormones, they are now recognized as broader metabolic and toxicological sensors and regulators, through their ability to discriminate small lipid molecules (Beato Metal. (1995) Cell 83:851-857; Kastner P et al. (1995) Cell 83:859-869; Chawla A et al. (2001) Science 294:1866-1870; Lazar MA (2017) J Clin. Invest 127:1123-1125). They primarily function as ligand-regulated transcription factors that control the expression of their target genes by interacting with specific DNA motifs in transcriptional promoters and enhancers, and recruiting other transcription factors and chromatin editors (McKenna NJ et al. (2002) Cell 108:465-474; Glass CK et al. (2010) Nat Rev. Immunol. 10:365-376). NRs are also eminently druggable molecules, because their action can be regulated through small molecules that are readily manipulable by chemical synthesis. NR-modulating small molecules mainly target the ligand-binding domain, but also interactions with transcriptional co-activators or with DNA. Currently, NRs are targets of some of the most widely used therapeutic agents, accounting for 13% of FDA approved drugs target NR family members (Overington JP et al. (2006) Nat Rev Drug Discov. 5:993-996). In fairness, these developments have been hampered by some of the complexities of NR action (many operate as heterodimers) or by uncertainty or redundancy of their ligands (a number of NRs remain categorized as orphans).

In the immune system, some NRs play important and pleiotropic functions. Most classically, corticosteroids acting via GR are major immunosuppressors. RORγ is a master regulator for the differentiation of several cell-types, perhaps best known for Th17, but also for Innate Like Lymphocytes and gdT cells. Retinoic acid acting via RARα or RARγ affects T cell differentiation (Hall JA et al. (2011) Immunity 35:13-22). NR4A1 is induced by and controls T cell activation. Even some “metabolic” NRs have been implicated in the function of particular immunocytes, like PPARγ in some Treg cells (Cipolletta D et al. (2012) Nature 486:549-553).

However, there is no comprehensive perspective on the role of NRs in immunologic cell-types. Even though many NRs are expressed in innate or adaptive immunocytes, their role remains often uncharted. Therefore, there is a need to understand the role of NRs in different immunologic cell-types, and as immunotherapy targets, to provide novel methods and therapies for major diseases, such as cancer, autoimmune disorders, or inflammatory diseases, among others.

### SUMMARY OF THE INVENTION

One aspect of the invention relates to a method for recovering from, treating, or preventing cancer in a subject in need thereof comprising administering an effective amount of: (a) an agent that modulates the level of, activity of, or expression of a nuclear receptor subfamily 2, group C, member 2 (Nr2c2), or fragment thereof, or nucleic acid encoding same; (b) an Nr2c2 variant, or fragment thereof, or nucleic acid encoding same; or (c) both (a) and (b); to the subject to thereby modulate T regulatory cell differentiation, function, activity, or maturation, or combination thereof, in the subject.

Another aspect of the invention relates to a method for treating or preventing a disorder associated with inflammation in a subject in need thereof comprising administering to the subject an effective amount of: (a) an agent that modulates the level of, activity of, or expression of Nr2c2, or fragment thereof, or nucleic acid encoding same; (b) an Nr2c2 variant, or fragment thereof, or nucleic acid encoding same; or (c) both (a) and (b); to the subject to thereby modulate T regulatory cell (Treg) differentiation, function, activity, or maturation, or combinations thereof, in the subject.

Another aspect of the invention relates to a method of modulating an inflammatory response in a subject in need thereof comprising administering to the subject an effective amount of: (a) an agent that modulates the level of, activity of, or expression of Nr2c2, or fragment thereof, or nucleic acid encoding same; (b) an Nr2c2 variant, or fragment thereof, or nucleic acid encoding same; or (c) both (a) and (b); to the subject to thereby modulate T regulatory cell differentiation, function, activity, or maturation, or combinations thereof, in the subject.

In some embodiments of any of the aforementioned methods, the agent inhibits, decreases, suppresses, reduces, knocks down, or depletes, the level of, actitvity of, or expression of the Nr2c2, or a fragment thereof, or a nucleic acid encoding same.

In some embodiments of any of the aforementioned methods, the Treg differentiation, function, activity, or maturation, or combinations theref, is blocked.

In some embodiments of any of the aforementioned methods, the agent activates, initiates, increases, or stimulates, the level of, actitvity of, or expression of the Nr2c2, or a fragment thereof, or a nucleic acid encoding same.

In some embodiments of any of the aforementioned methods, the Treg differentiation, function, activity, or maturation, or combinations thereof, is enhanced.

In some embodiments of any of the aforementioned methods, the agent inhibits, decreases, suppresses, reduces, knock downs, or depletes the level of, actitvity of, or expression of the Nr2c2, or homologs thereof, as set forth in Table 1, Table 2, or combinations thereof.

In some embodiments of any of the aforementioned methods, the agent is an antagonist of Nr2c2.

In some embodiments of any of the aforementioned methods, the agent activates, initiates, increases, or stimulates the level of, actitvity of, or expression of the Nr2c2, or homologs thereof, as set forth in Table 1, Table 2, or combinations thereof.

In some embodiments of any of the aforementioned methods, the agent is an agonist of Nr2c2.

In some embodiments of any of the aforementioned methods, the agonist of Nr2c2 is a polyunsaturated fatty acid (PUFA), or metabolite thereof.

In some embodiments of any of the aforementioned methods, the PUFA is selected from omega-3 fatty acid or omega-6 fatty acid.

In some embodiments of any of the aforementioned methods, the PUFA metabolite is selected from the group consisting of 15-hydroxyeico-satetraonic acid (15-HETE), 13-hydroxy octa-deca dieonic acid (13-HODE), and thiazolidinedione (TZD)-rosiglitazone.

In some embodiments of any of the aforementioned methods, the agonist of Nr2c2 is a retinoid.

In some embodiments of any of the aforementioned methods, the retinoid is an all-trans-retinoic acid, retinol (ATRA).

In some embodiments of any of the aforementioned methods, the agonist of Nr2c2 is a keto mycolic acid from Mycobacterium tuberculosis cell wall lipids.

In some embodiments of any of the aforementioned methods, the agonist of Nr2c2 is γ-linoleic acid.

In some embodiments of any of the aforementioned methods, the Nr2c2 variant, or fragment thereof, or nucleic acid encoding same, comprises at least one substitution, mutations, insertion, deletion, or combination thereof, in Nr2c2 as set forth in Table 1, Table 2, or combinations thereof.

In some embodiments of any of the aforementioned methods, the Nr2c2 variant, or fragment thereof, or nucleic acid encoding same, comprises at least two, three, four, five, six, seven, eight, nine, ten, or more substitution, mutations, insertion, deletion, or combiantions thereof, in Nr2c2 as set forth in Table 1, Table 2, or combinations thereof.

In some embodiments of any of the aforementioned methods, the Nr2c2 variant, or fragment thereof, or nucleic acid encoding same, is biologically inactive or functionally defective.

In some embodiments of any of the aforementioned methods, the Treg maturation, differentiation, activity, or function, or combination thereof, is inhibited, decreased, suppressed, reduced, knocked down, or depleted.

In some embodiments of any of the aforementioned methods, the Nr2c2 variant, or fragment thereof, or nucleic acid encoding same, is biologically active or functionally active.

In some embodiments of any of the aforementioned methods, the Treg maturation, differentiation, activity, or function, or combination thereof, is activated, initiated, increased, or stimulated.

In some embodiments of any of the aforementioned methods, the agent knocks down, reduces, eliminates, or decreases Nr2c2 gene levels, expression levels, or both.

In some embodiments of any of the aforementioned methods, the agent is selected from siNA, Clustered Regularly Interspaced Short Palindromic Repeats-Caspase 9 (CRISPR/Cas9), Transcription activator-like effector nucleases (TALEN), or zinc-finger nuclease (ZFN).

In some embodiments of any of the aforementioned methods, inflammation is decreased.

In some embodiments of any of the aforementioned methods, an inflammatory response is depressed or suppressed.

In some embodiments of any of the aforementioned methods, the cancer is selected from the group consisting of acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hypopharyngeal cancer, intraocular melanoma, islet cell tumors (endocrine pancreas), Kaposi sarcoma, Langerhans cell histiocytosis, laryngeal cancer, leukemia, lung cancer, non-small cell lung cancer, small cell lung cancer, Hodgkin lymphoma, lymphoma, medulloblastoma, medulloepithelioma, melanoma, mesothelioma, mouth cancer, multiple myeloma, nasopharyngeal cancer, neuroblastoma, non-Hodgkin lymphoma, oral cancer, oropharyngeal cancer, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm, pleuropulmonary blastoma, primary central nervous system lymphoma, prostate cancer, rectal cancer, renal cell (kidney) cancer, rhabdomyosarcoma, salivary gland cancer, sarcoma, Ewing sarcoma family of tumors, sarcoma, Sezary syndrome, skin cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

In some embodiments of any of the aforementioned methods, the method of claim 3, wherein the disorder associated with inflammation is selected from the group consisting of: septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacteria and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, and pulmonary inflammation.

In some embodiments of any of the aforementioned methods, the agent or Nr2c2 variant is administered to the subject at a dose of between 0.5-5 grams per day.

In some embodiments of any of the aforementioned methods, the agent or the Nr2c2 variantis administered in a pharmaceutically effective amount.

In some embodiments of any of the aforementioned methods, the pharmaceutically effective amount is provided as a pharmaceutical composition in combination with a pharmaceutically-acceptable excipient, diluent, or carrier.

In some embodiments of any of the aforementioned methods, the a) agent is administered simultaneously as the Nr2c2 variant, or fragment thereof, or nucleic acid encoding same; b) agent is administered in combination with Nr2c2 variant, or fragment thereof, or nucleic acid encoding same; c) agent is administered prior to administering the Nr2c2 variant, or fragment thereof, or nucleic acid encoding same; or d) agent is administered subsequently to administering the Nr2c2 variant, or fragment thereof, or nucleic acid encoding same.

In some embodiments of any of the aforementioned methods, the subject is a mammal or non-mammal.

In some embodiments of any of the aforementioned methods, the subject is a human.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 depicts a schematic representation of the CRISPR library screen for NR function in immune cell differentiation and homeostasis.

FIG. 2 contains two panels, 2A and 2B, depicting a screen for nuclear receptors in Treg. 2A is the heatmap showing the count ratio of sgRNAs targeting each gene in Treg versus Tconv cells in each bone marrow chimera mouse. All sgRNAs targeting the same gene were pooled for counting in each mouse. Gray blocks represented insufficient representation in the host mouse and being filtered out during analysis. 2B is a graph wherein the x axis shows the mean ratio of sgRNAs targeting each gene in Treg verus Tconv, and the y axis shows the one-sample test P value.

FIG. 3 contains five panels, 3A-3E, depicting phenotypic alterations in Nr2c2-deficient Tregs. 3A depicts a schematic representation of validation the role of Nr2c2 in Tregs via CRISPR knockout. LSK cells sorted from Cas9-expressing mice are delivered via lentivirus with non-targeting sgRNA (sgCtrl) and sgRNA targeting Nr2c2 (sgNr2c2), respectively. sgCtrl and sgNr2c2 are labeled with GFP and RFP reporter fluorescent protein, respectively. LSK cells are then mixed together to reconstitute lethally irradiated host mice. After 10 weeks, the GFP immune cells represent WT or Ctrl cells, including Tregs. While RFP-positive ones represent Nr2c2-deficient cells, including Tregs. 3B shows the Treg percentage among all TCRbeta and CD4 double positive cells in BMC mice, with GFP-sgCtrl and RFP-sgNr2c2, or GFP-sgFoxp3 and RFP-sgCtrl, respectively. Flow cytometry plots show the gating strategy, and histograms are statistic results. 3C, shows the surface marker (CD44 and CD62L) staining on Tregs in BMC mice with GFP-sgCtrl and RFP-sgNr2c2, or GFP-sgCtrl and RFP-sgNr2c1, respectively. Flow cytometry plots show the gating strategy, and histograms are statistic results. 3D shows the surface maker PD-1 staining on Tregs with sgCtrl and sgNr2c2 in BMC mice, respectively. Flow cytometry plots show the gating strategy, and histograms are statistic results.

FIG. 4 contains two panels, 4A and 4B, depicting transcriptome alterations in Nr2c2-deficient Tregs. Volcano plots comparing transcriptomes of Tregs (4A) or Tconvs (4B) with sgCtrl or sgNr2c2 from BMC mice. The x axis shows the fold change of expression value in cells with sgCtrl versus those with sgNr2c2, and the y axis shows the p-value. Activated versus resting Treg signature genes (from GSE71309) are highlighted in red (up regulated) or blue (down regulated). Values at the bottom represent the number of genes from each signature differentially expressed in one population or the other. P values are determined by Chi-squared t-test.

FIG. 5 contains four panels, 5A-5D, depicting increased oxidative phosphorylation in Nr2c2-deficient Tregs. Volcano plot comparing transcritomes of Tregs with sgCtrl or sgNr2c2 from BMC mice in 5A, or Tconvs in 5D. Gene Ontology gene sets of oxidative phosphorylation (term GO:0006119) are highlighted in red. 5B, electron transport chain in mitochondria and complexes involved in oxidative phosphorylation. The values at the bottom represent numbers of complex protein coded by mitochondrial DNA or nuclear DNA. 5C, heatmap shows the relative expression levels (lower in blue and higher in red) of indicated oxidative phorphorylation complex proteins in Tregs with sgCtrl or sgNr2c2 from BMC mice.

Note that for every figure containing a histogram, the bars from left to right for each discreet measurement correspond to the figure boxes from top to bottom in the figure legend as indicated.

FIG. 6 shows underexpression of metabolic pathways in rTregs from Nr2c2-decificient rTregs. Bone Marrow Chimera mice were generated with a mixture of bone marrow stem cells of Cas9 transgenic mice transduced with sgRNAs targeting Nr2c2 or control sgRNA. Resting Tregs (rTreg) with non-targeting control sgRNA (sgCtrl) and Nr2c2-targeting sgRNA (sgNr2c2) were sorted from spleens of the resulting bone-marrow chimera mice after 8 weeks, and profiled by RNAseq. Gene enrichment analysis (GSEA) was performed on the data. The results show that multiple mitochondrial respiration pathways are differentially represented in Ctrl vs Nr2c2-targeted cells.

FIG. 7 shows representative GSEA plots, which show that oxidative phosphorylation, electron transport chain, mitochondrial respiratory chain complex assembly, and multi-organism metabolic process pathways were deficient in Nr2c2-deficient CRISPR-KO Tregs.

FIG. 8 shows Nr2c2-KO Treg homeostasis/survival ability is lower in vivo. Tregs with non-targeting control sgRNA or Nr2c2-targeting sgRNA were sorted from spleens and lymph nodes of Bone Marrow Chimera mice, and then transferred into Rag1−/− mice together with a bolus of congenic naïve CD4+ T cells. The results show a decreased capacity for survival of Nr2c2-deficient Treg cells.

FIG. 9 shows Nr2c2-KO Treg homeostasis/survival ability is lower in vivo. Mature Tregs were sorted from wild type mice and transduced with non-targeting control (RFP vector) or Nr2c2-targeting sgRNA (GFP vector) via retroviral infection in vitro. Then Tregs transduced with RFP-sgCtrl and GFP-sgNr2c2 were mixed and co-transferred into Rag1−/− mice together with splenocytes from wild type mice. Rag1−/− mice were analyzed at indicated time post transfer. The results show that, in a competitive setting with wild-type Tregs, Nr2c2 deficient Tregs have a reduced fitness.

FIG. 10 shows Nr2c2-deficient Tregs have less mitochondrial mass and lower mitochondrial respiratory ability. Tregs with non-targeting control sgRNA or Nr2c2-targeting sgRNA from spleens and lymph nodes of Bone Marrow Chimeras mice prepared as above were analyzed by flow cytometry using dyes that reveal Mitochondrial mass (MitoTracker Deep Red) and activity (DiIC1(5)). The results show that Nr2c2 is necessary to support full mitochondrial load and activity, concordant with the gene expression profiling.

### DETAILED DESCRIPTION OF THE INVENTION

### 1. Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximate”) one particular value, and/or to “about” (or “approximate”) another particular value. When such a range is expressed, another embodiment includes from the one particular value and/or to the other particular value. Similarly, when values are expressed as approximations, by use of the antecedent “about” or “approximate” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that is “less than or equal to the value” or “greater than or equal to the value” possible ranges between these values are also disclosed, as appropriately understood by the skilled artisan. For example, if the value “10” is disclosed the “less than or equal to 10” as well as “greater than or equal to 10” is also disclosed.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e. g. “such as”) provided herein is intended merely to better illuminate the invention and does not pose a limitation on the scope of the invention otherwise claimed.

Units, prefixes, and symbols are denoted in their Système International de Unites (SI) accepted form. Unless otherwise indicated, nucleic acids are written left to right in 5′ to 3′ orientation; amino acid sequences are written left to right in amino to carboxy orientation.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise. As used herein, the term “about” refers to a range of values of plus or minus 10% of a specified value. For example, the phrase “about 200” includes plus or minus 10% of 200, or from 180 to 220, unless clearly contradicted by context.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

An agent that is an “antagonist” if said agent down regulates or blocks the biological function of the cell surface receptor. As used herein, an agent which is an “antagonist” includes agents that bind or otherwise interfere with ligands of cell surface receptor (e.g., nuclear receptor) thereby blocking the ability of the ligand to bind to the cell surface receptor and down-regulate or prevent the biological function of the cell surface receptor.

An agent that is an “agonist” if said agent upregulates or increases the biological function of the cell surface receptor (e.g., nuclear receptor).

As used herein, administration “in combination” refers to both simultaneous and sequential administration of two or more compositions. Concurrent or combined administration, as used herein, means that two or more compositions are administered to a subject either (a) simultaneously, or (b) at different times during the course of a common treatment schedule. In the latter case, the two or more compositions are administered sufficiently close in time to achieve the intended effect.

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer is generally associated with uncontrolled cell growth, invasion of such cells to adjacent tissues, and the spread of such cells to other organs of the body by vascular and lymphatic menas. Cancer invasion occurs when cancer cells intrude on and cross the normal boundaries of adjacent tissue, which can be measured by assaying cancer cell migration, enzymatic destruction of basement membranes by cancer cells, and the like. In some embodiments, a particular stage of cancer is relevant and such stages can include the time period before and/or after angiogenesis, cellular invasion, and/or metastasis. Cancer cells are often in the form of a solid tumor, but such cells may exist alone within an animal, or may be a non-tumorigenic cancer cell, such as a leukemia cell. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present invention is used in the treatment, diagnosis, and/or prognosis of melanoma and its subtypes.

As used herein, the terms “effective amount,” “effective dose,” “sufficient amount,” “amount effective to,” “therapeutically effective amount,” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting or transit that can be associated with the administration of the pharmaceutical composition. With respect to “effective amount,” “effective dose,” “sufficient amount,” “amount effective to,” or “therapeutically effective amount” of a probiotic microorganism, the dosing range varies with the probiotic microorganism used, the route of administration and the potency of the particular probiotic microorganism.

The terms “enhance”, “promote” or “stimulate” in terms of an immune response includes an increase, facilitation, proliferation, for example a particular action, function or interaction associated with an immune response.

The terms “individual,” “subject,” “host,” and “patient,” used interchangeably herein, refer to a mammal, including, but not limited to, murines, simians, felines, canines, equines, bovines, mammalian farm animals, mammalian sport animals, and mammalian pets and humans. Preferred is a human.

“Immune-related disease” means a disease in which the immune system is involved in the pathogenesis of the disease. Subsets of immune-related diseases are autoimmune diseases. Autoimmune diseases include, but are not limited to, rheumatoid arthritis, myasthenia gravis, multiple sclerosis, psoriasis, systemic lupus erythematosus, autoimmune thyroiditis (Hashimoto's thyroiditis), Graves' disease, inflammatory bowel disease, autoimmune uveoretinitis, polymyositis, and certain types of diabetes. In view of the present disclosure, one skilled in the art can readily perceive other autoimmune diseases treatable by the compositions and methods of the present invention.

As used herein, the term “immune response” includes T cell mediated and/or B cell mediated immune responses. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells, e.g., macrophages.

Examples of diseases or conditions wherein enhancement of a protective immune response is desired includes, but are not limited to viral, pathogenic, protozoal, bacterial, or fungal infections and cancer.

Viral infectious diseases include human papilloma virus (HPV), hepatitis A Virus (HAV), hepatitis B Virus (HBV), hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, influenza virus, Hepatitis A and B, FIV, lentiviruses, pestiviruses, West Nile Virus, measles, smallpox, cowpox, ebola, coronavirus, retrovirus, herpesvirus, potato S virus, simian Virus 40 (SV40), Mouse Mammary Tumor Virus (MMTV) promoter, Moloney virus, ALV, Cytomegalovirus (CMV), Epstein Barr Virus (EBV), or Rous Sarcoma Virus (RSV). In addition, bacterial, fungal and other pathogenic diseases are included, such as Aspergillus, Brugia, Candida, Chikungunya, Chlamydia, Coccidia, Cryptococcus, Dengue, Dirofilaria, Gonococcus, Histoplasma, Leishmania, Mycobacterium, Mycoplasma, Paramecium, Pertussis, Plasmodium, Pneumococcus, Pneumocystis, P. vivax in Anopheles mosquito vectors, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Clostridium tetani, Clostridium botulinum; or, a fungus, such as, e.g., Paracoccidioides brasiliensis; or other pathogen, e.g., Plasmodium falciparum. Also included are National Institute of Allergy and Infectious Diseases (NIAID) priority pathogens. These include Category A compositions, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B compositions, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain O157:H7, Vibrio cholerae, Cryptosporidium parvum; Category C compositions, such as nipah virus, hantaviruses, yellow fever in Aedes mosquitoes, and multidrug-resistant tuberculosis; helminths, such as Schistosoma and Taenia; and protozoa, such as Leishmania (e.g., L. mexicana) in sand flies, Plasmodium, Chagas disease in assassin bugs.

Other bacterial pathogens include, but are not limited to, bacterial pathogenic gram-positive cocci, which include but are not limited to: pneumococci; staphylococci; and streptococci. Pathogenic gram-negative cocci include: meningococci; and gonococci. Pathogenic enteric gram-negative bacilli include: enterobacteriaceae; pseudomonas, acinetobacteria and eikenella; melioidosis; salmonella; shigellosis; hemophilus; chancroid; brucellosis; tularemia; yersinia (pasteurella); streptobacillus moniliformis and spirilum; listeria monocytogenes; erysipelothrix rhusiopathiae; diphtheria; cholera; anthrax; and donovanosis (granuloma inguinale). Pathogenic anaerobic bacteria include; tetanus; botulism; other clostridia; tuberculosis; leprosy; and other mycobacteria. Pathogenic spirochetal diseases include: syphilis; treponematoses: yaws, pinta and endemic syphilis; and leptospirosis. Other infections caused by higher pathogen bacteria and pathogenic fungi include: actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include rickettsial and rickettsioses. Examples of mycoplasma and chlamydial infections include: mycoplasma pneumoniae; lymphogranuloma venereum; psittacosis; and perinatal chlamydial infections. Pathogenic protozoans and helminths and infections eukaryotes thereby include: amebiasis; malaria; leishmaniasis; trypanosomiasis; toxoplasmosis; pneumocystis carinii; giardiasis; trichinosis; filariasis; schistosomiasis; nematodes; trematodes or flukes; and cestode (tapeworm) infections. While not a disease or condition, enhancement of a protective immune response is also beneficial in a vaccine or as part of a vaccination regimen as is described herein.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. As used herein, “inhibition of Nr2c2 nucleic acid expression” or “inhibition of Nr2c2 gene expression” includes any decrease in expression or protein activity or level of the Nr2c2 nucleic acid or protein encoded by the Nr2c2 nucleic acid. The decrease may be of at least 30%, 40%, 50%, 60%, 70%, 80%, 90%, 95% or 99% or more as compared to the expression of a target nucleic acid or the activity or level of the protein encoded by a target nucleic acid which has not been targeted by an RNA interfering agent. “Inhibition of Nr2c2 nucleic acid expression” or “inhibition of Nr2c2 gene expression” may be mediated by any of the gene editing methods known in the art, including but not limited to siNA, Clustered Regularly Interspaced Short Palindromic Repeats-Caspase 9 (CRISPR/Cas9), Transcription activator-like effector nucleases (TALEN), or zinc-finger nuclease (ZFN).

As used herein, the term “modulate” includes up-regulation and down-regulation, e.g., enhancing or inhibiting an immune response, inflammatory response, or Treg differentiation, maturation, activity, or function. The term “modulate” when used with regard to modulation of a receptor includes up-regulation or down-regulation of the biological activity associated with that receptor when the receptor is activated, for example, by its ligand (e.g., agonist) or inhibited, for example, with an antagonist, or a blocking antibody.

As used herein, “Nr2c2” or “NR2C2” refers to nuclear receptor subfamily 2, group C, member 2. Nr2c2 may also be referred to as nuclear hormone receptor TR4 testicular nuclear recptor 4, TR4, TAK1, TR2R1, hTAK1. Nr2c2 is a member of the nuclear hormone receptor family that act as ligand-activated transcription factors. The proteins have an N-terminal transactivation domain, a central DNA-binding domain with 2 zinc fingers, and a ligand-binding domain at the C terminus. The activated receptor/ligand complex is translocated to the nucleus where it binds to hormone response elements of target genes (Yoshikawa, T et al. (1996) i Genomics 35: 361-366). Chang et al. cloned NR2C2, or TR4, using degenerate PCR on RNA from the supraoptic nucleus of the brain with primers based on the conserved DNA-binding domain of these genes (Chang et al. (1994) Proc. Nat. Acad. Sci. 91: 6040-6044). They isolated TR4 cDNAs from both human and rat libraries. The cDNAs encode a predicted 615-amino acid human protein and a 596-amino acid rat protein that are 98% identical. The TR4 sequence is similar to that of the TR2 orphan receptor (Chang et al. (1994) Proc. Nat. Acad. Sci. 91: 6040-6044). Together they appear to form a distinct subfamily. Hirose et al. cloned the TR4 gene, which they designated TAK1, from a human lymphoblastoma cDNA library (Hirose et al. (1994) Molec. Endocr. 8: 1667-1680). They stated that the predicted protein is 596 amino acids long. On SDS-PAGE, TR4 migrated as a 65-kD protein. Using Northern blot analysis, Hirose et al. (1994) found that TR4 is expressed as a 9.4-kb mRNA in many tissues, and as a 2.8-kb mRNA primarily in testis. The two transcripts appeared to differ in the length of the 3-prime untranslated region. In mouse and rat testis, TR4 was expressed most abundantly in spermatocytes. Yoshikawa et al. used RT-PCR to show that in rat and human, 2 isoforms (with and without a 19 codon exon) could be detected; in human, they were both expressed widely in tissues including brain, placenta, and ovary (Yoshikawa et al. (1996) Genomics 35: 361-366). However, in the PA1 human ovarian cancer cell line only the short form was detected. The difference in cDNA length is a consequence of alternative splicing. Nakajima et al. stated that TAK1 binds as a homodimer to direct repeats of the consensus sequence AGGTCA (Nakajima et al. (2004) Nucleic Acids Res. 32: 4194-4204). They identified TIP27 (JAZF1; 606246) as a repressor of TAK1 transcriptional activity. Yeast and mammalian 2-hybrid analyses revealed that TIP27 interacted with TAK1, but not with other nuclear receptors, either in the presence or absence of their respective ligands. Protein pull-down and immunoprecipitation analyses confirmed the interaction between TAK1 and TIP27. Deletion analysis revealed that an N-terminal domain of TIP27 interacted with a portion of the ligand-binding domain of TAK1. Reporter gene assays showed that TIP27 repressed TIP1 transcriptional activity in a dose-dependent manner. TIP27 did not interfere with TIP1 homodimerization or with binding of TIP1 to DNA, suggesting that interaction of TIP27 with TAK1 may inhibit recruitment of a coactivator. Yoshikawa et al. mapped the human NR2C2 gene to chromosome 3 using a somatic cell yybrid panel and refined the localization to 3p25 by fluorescence in situ hybridization (Yoshikawa et al. (1996) Genomics 35: 361-366). Collins et al. found that Nr2c2-null mice were born at lower than Mendelian ratios, with a significantly lower proportion of female than male knockout mice (Collins et al. Proc. Nat. Acad. Sci. 101: 15058-15063). A growth defect was apparent early in postnatal life in affected mice, and their fertility was greatly reduced. Additionally, female mice lacking Nr2c2 exhibited behavioral abnormalities, including defects in maternal behavior.

Independently, Mu et al. found that Tr4−/− mice were smaller than wildtype. Tr4−/− mice exhibited varying degrees of behavioral defects, such as hypersensitivity to environmental stimuli (Mu et al. (2004) Molec. Cell. Biol. 24:5887-5899). Male fertility was reduced in Tr4−/− mice and was associated with delayed spermatogenesis and decreased sperm production. Chen et al. reported that behavioral abnormalities in Tr4−/− mice included mild trembling, unsteady gait, hyperreactivity upon manipulation, hind limb grasping, decreased tendency to explore surroundings, and impaired motor coordination and balance (Chen et al. (2005) Molec. Cell. Biol. 25: 2722-2732). Histologic examination of postnatal Tr4−/− cerebellum revealed gross abnormalities in foliation, with loss of lobule VII in the anterior vermis. Laminations of Tr4−/− cerebellar cortex were abnormal, and Purkinje cells showed aberrant dendritic arborization and loss of calbindin (see CALB1, 114050) staining. Developing Tr4−/− cerebellum exhibited reduced expression of genes involved in cerebellar morphologic development.

Nr2c2 members share a common structural organization with a central well-conserved DNA binding domain (DBD), a variable N-terminal domain, a non-conserved hinge, and a C-terminal ligand binding domain (LBD). The superfamily contains not only receptors for known ligands, but also orphan receptors for which ligands do not exist or have not been identified. The members of this family include receptors of steroids, thyroid hormone, retinoids, cholesterol by-products, lipids and heme. The conserved DBD is a DNA-binding domain of nuclear receptors and is composed of two C4-type zinc fingers. Each zinc finger contains a group of four Cys residues which co-ordinates a single zinc atom. It interacts with a specific DNA site upstream of the target gene and modulates the rate of transcriptional initiation. Nuclear receptors form a superfamily of ligand-activated transcription regulators, which regulate various physiological functions, from development, reproduction, to homeostasis and metabolism in animals (metazoans). The family contains not only receptors for known ligands but also orphan receptors for which ligands do not exist or have not been identified. Most nuclear receptors bind as homodimers or heterodimers to their target sites, which consist of two hexameric half-sites. Specificity is determined by the half-site sequence, the relative orientation of the half-sites and the number of spacer nucleotides between the half-sites. However, a growing number of nuclear receptors have been reported to bind to DNA as monomers.

Representative Nr2c2 nucleotide and amino acid sequences are set forth below. The nucleotide and amino acid sequence information for the aforementioned nucleic acids and proteins are well known in the art and readily available on publicly available databases, such as the National Center for Biotechnology Information (NCBI). For example, exemplary nucleotide sequences derived from publicly available sequence databases are provided below in Table 1. Exemplary amino acid sequences derived from publicly available sequence databases are provided below in Table 2.

TABLE 1
Nr2c2 (nucleotide sequences)

NM_003298.4 Homo sapiens
(SEQ ID NO: 1) (1848 bp)
atgaccagcc cctccccacg catccagata atctccaccg
actctgctgt agcctcacct cagcgcattc agggctctga
acctgcctct ggcccattga gtgttttcac atctttgaac
aaagagaaga ttgtcacaga ccagcagaca ggacagaaaa
tccagatagt caccgcagtg gacgcctccg gatcccccaa
acagcagttc atcctgacca gcccagatgg agctggaact
gggaaggtga tcctggcttc cccagagaca tccagcgcca
agcaactcat attcaccacc tcagacaacc tcgtccctgg
caggatccag attgtcacgg attctgcctc tgtggagcgt
ttactgggga agacggacgt ccagcggccc caggtggtag
agtactgtgt ggtctgtggc gacaaagcct ccggccgtca
ctatggggct gtcagttgtg aaggttgcaa aggtttcttc
aaaaggagtg tgaggaaaaa tttgacctac agctgccgga
gcaaccaaga ctgcatcatc aataaacatc accggaaccg
ctgtcagttt tgccggctga aaaaatgctt agagatgggc
atgaaaatgg aatctgtgca gagtgaacgg aagcccttcg
atgtgcaacg ggagaaacca agcaattgtg ctgcttcaac
tgagaaaatc tatatccgga aagacctgag aagtcccctg
atagctactc ccacgtttgt ggcagacaaa gatggagcaa
gacaaacagg tcttcttgat ccagggatgc ttgtgaacat
ccagcagcct ttgatacgtg aggatggtac agttctcctg
gccacggatt ctaaggctga aacaagccag ggagctctgg
gcacactggc aaatgtagtg acctcccttg ccaacctaag
tgaatctttg aacaacggtg acacttcaga aatccagcca
gaggaccagt ctgcaagtga gataactcgg gcatttgata
ccttagctaa agcacttaat accacagaca gctcctcttc
tccaagcttg gcagatggga tagacaccag tggaggaggg
agcatccacg tcatcagcag agaccagtcg acacccatca
ttgaggttga aggccccctc ctttcagaca cacacgtcac
atttaagcta acaatgccca gtccaatgcc agagtacctc
aacgtgcact acatctgtga gtctgcatcc cgtctgcttt
tcctctcaat gcactgggct cggtcaatcc cagcctttca
ggcacttggg caggactgca acaccagcct tgtgcgggcc
tgctggaatg agctcttcac cctcggcctg gcccagtgtg
cccaggtcat gagtctctcc accatcctgg ctgccattgt
caaccacctg cagaacagca tccaggaaga taaactttct
ggtgaccgga taaagcaagt catggagcac atctggaagc
tgcaggagtt ctgtaacagc atggcgaagc tggatataga
tggctatgag tatgcatacc ttaaagctat agttctcttt
agccccgatc atccaggttt gaccagcaca agccagattg
aaaaattcca agaaaaggca cagatggagt tgcaggacta
tgttcagaaa acctactcag aagacaccta ccgattggcc
cggatcctcg ttcgcctgcc ggcactcagg ctgatgagct
ccaacataac agaagaactt ttttttactg gtctcattgg
caatgtttcg atagacagca taatccccta catcctcaag
atggagacag cagagtataa tggccagatc accggagcca
gtctatag
XM_001158643.5 P. troglodytes
(SEQ ID NO: 2) (1848 bp)
atgaccagcc cctccccacg catccagata atctccaccg
actctgctgt agcctcacct cagcgcattc agggctctga
acctgcctct ggcccattga gtgttttgac atctttgaac
aaagagaaga ttgtcacaga ccagcagaca ggacagaaga
tccagatagt caccgcagtg gacgcctccg gatcccccaa
acagcagttc atcctgacca gcccagatgg agctggaact
gggaaggtga tcctggcttc cccagagaca tccagcgcca
agcaactcat attcaccacc tcagacaacc tcgtccctgg
caggatccag attgtcacgg attctgcctc tgtggagcgt
ttactgggga agacggatgt ccagcggccc caggtggtag
agtactgtgt ggtctgtggc gacaaagcct ccggccgtca
ctatggggct gtcagttgtg aaggttgcaa aggtttcttc
aaaaggagtg tgaggaaaaa tttgacctac agctgccgga
gcaaccaaga ctgcatcatc aataaacatc accggaaccg
ctgtcagttt tgccggctga aaaaatgctt agagatgggc
atgaaaatgg aatctgtgca gagtgaacgg aagcccttcg
atgtgcaacg ggagaaacca agcaattgtg ctgcttcaac
tgagaaaatc tatatccgga aagacctgag aagtcccctg
atagctactc ccacgttcgt ggcagacaaa gatggagcaa
gacaaacagg tcttcttgat ccagggatgc ttgtgaacat
ccagcagcct ttgatacgtg aggatggtac agttctcctg
gccacggatt ctaaggctga aacaagccag ggagctctgg
gcacactggc aaatgtagtg acctcccttg ccaacctaag
tgaatctttg aacaacggtg acacttcaga aatccagcca
gaggaccagt ctgcaagtga gataactcgg gcatttgata
ccttagctaa agcacttaat accacagaca gctcctcttc
tccaagcttg gcagatggga tagacaccag cggaggaggg
agcatccacg tcatcagcag agaccagtcg acacccatca
ttgaggttga aggccccctc ctttcagaca cacacgtcac
atttaagcta acaatgccca gtccaatgcc agagtacctc
aacgtgcact acatctgtga gtctgcatcc cgtctgcttt
tcctctcaat gcactgggct cggtcaatcc cagcctttca
ggcacttggg caggactgca acaccagcct ggtgcgggcc
tgctggaatg agctcttcac cctcggcctg gcccagtgtg
cccaggtcat gagtctctcc accatcctgg ctgccattgt
caaccacctg cagaacagca tccaggaaga taaactttcc
ggtgaccgga taaagcaagt catggagcac atctggaagc
tgcaggagtt ctgtaacagc atggcgaagc tggatataga
tggctatgag tatgcatacc ttaaagctat agttctcttt
agccccgatc atccaggttt gaccagcaca agccagattg
aaaaattcca agaaaaggca cagatggagt tgcaggacta
tgttcagaaa acctactcag aagacaccta ccgattggcc
cggatcctcg ttcgcctgcc ggcactcagg ctgatgagct
ccaacataac agaagaactt ttttttactg gtctcattgg
caatgtttcg atagacagca taatccccta catcctcaag
atggagacag cagagtataa tggccagatc accggagcca
gtctatag
XM_002798704.1 M. mulatta
(SEQ ID NO: 3) (1947 bp)
atggctacaa atatggaggg gctggttcag cacagagtgg
ggacccagca ggtggctgag gtaacacgta cacagacctc
tcggccggaa tctccaggga tgaccagccc ctccccacgc
atccagataa tctccaccga ctctgctgta gcctcacctc
agcgcattca gggctctgaa cctgcctctg gcccattgag
tgttttcacc tctttgaaca aagagaagat tgtcacagac
cagcagacag gacagaagat ccagatagtc accgcagtgg
acgcctccgg atcccctaaa caacagttca tcctgaccag
cccagatgga gctggaactg ggaaggtgat cctggcttcc
ccagagacat ccagcgccaa gcaactcata tttaccacct
cagacaacct cgtccctggc aggatccaga ttgtcacgga
ttctgcctct gtggagcgtt tactggggaa gacggacgtc
cagcggcccc aggtggtaga gtactgtgtg gtctgtggcg
acaaagcctc cggccgtcac tatggggctg tcagttgtga
aggttgcaaa ggtttcttca aaaggagcgt gaggaaaaat
ttgacctaca gctgtcggag caaccaagac tgcatcatca
ataaacatca ccggaaccgc tgtcagtttt gccggctgaa
aaaatgctta gagatgggca tgaaaatgga atccgtacag
agtgaacgga agccctttga tgtgcaacgg gagaaaccaa
gcaattgtgc tgcttcaact gagaaaatct atatccggaa
agacctgaga agtcccctga tagccactcc cacgtttgtg
gcagacaaag acggagcgag acaaacaggt cttcttgatc
caggaatgct tgtgaacatc cagcagcctt tgatacgtga
ggatggtaca gttcttctgg ccacggattc caaggctgaa
acaagccagg gagctctggg cacactggca aatgtagtga
cctcgcttgc caacctaagt gaatctttga acaacggtga
cacttcagaa atccaaccag aggaccagtc tgcaagtgag
attactcggg catttgatac cttagctaaa gcacttaata
ccacagacag ctcctcctct ccaagcctgg cagatggaat
agacaccagt ggaggaggaa gcatccacgt catcagccga
gaccagtcga cacccatcat tgaggttgaa ggccccctcc
tttcagacac acacgtcaca tttaagctaa caatgcccag
cccaatgcca gagtacctca acgtgcacta catctgtgag
tctgcatccc gtctgctttt cctctcaatg cactgggctc
ggtcaatccc agcctttcag gcacttgggc aggactgcaa
caccagcctg gtgcgggcct gctggaatga gctcttcacc
ctcggcctgg cccagtgtgc ccaggtcatg agtctctcca
ccatcctggc tgccatcgtc aaccacctgc agaacagcat
ccaggaagat aaactttctg gtgaccggat aaagcaagtc
atggagcaca tctggaagct gcaggagttc tgtaacagca
tggcgaagct ggatatagat ggctatgagt atgcatacct
taaagctata gttctctttn tttcagatca tccaggtttg
accagcacaa gccagattga aaaattccaa gaaaaggcac
agatggagtt gcaggactat gttcagaaaa cctactcgga
agacacctac cgattggccc ggatcctcgt tcgcctgccg
gcactcaggc tgatgagctc caacataaca gaagaacttt
tttttactgg cctcattggc aatgtttcga tagacagcat
aatcccctac atcctcaaga tggagacagc agagtataat
ggccagatca ccggagccag tctatag
XM_005632118.3 C. lupus
(SEQ ID NO: 4) (1890 bp)
atggctacaa atatggaggg gctggttcag cacagagtgg
ggacccagca ggtggctgag gtaccacgga cacagacctc
tcggccggaa tctccaggga tgaccagccc ctccccgcgc
atccagataa tctccaccga ctctgcggta gcttcacctc
agcgaattca gattgtgaca gaccagcaga caggacagaa
gatccagata gtcaccgcag tggacgcctc cggatccccc
aagcagcagt tcatcctgac cagcccagat ggagctggaa
ctgggaaggt gatcctggct tccccggaga catccagtgc
taagcagctc atattcacca cctcggacaa ccttgtccct
ggcaggatcc agatcgtcac ggactctgct tctgtggagc
gcttgctggg taaggccgac gtccagcggc cacaggtggt
agagtactgt gtcgtctgtg gcgacaaagc ctctggtcgt
cactatgggg ctgtcagttg tgaaggttgc aaaggtttct
ttaaaaggag tgtaaggaaa aatctgacct acagctgccg
gagcaaccaa gactgtatca tcaataaaca ccaccggaac
cgctgtcagt tttgccggct gaaaaaatgc ttagagatgg
gcatgaagat ggaatctgtg cagagtgaac ggaagccctt
tgatgtacaa cgggagaaac caagcaattg tgctgcttca
actgagaaaa tttatatccg gaaggacttg agaagtcctc
tgatagctac tcccacgttt gtggcagata aagatggagc
aagacaaaca ggtcttcttg atccagggat gcttgtcaac
atccaacagc ctttgatacg tgaggatggt acagttctcc
tggccacgga ttccaaggct gaaacaagcc agggagctct
gggcacactg gccaatgtag taacctccct cgccaactta
agtgaatccc ttaacaatgg tgatgcttcg gaaatgcagc
cagaggacca gtctgcgagt gagattactc gggcatttga
taccttagct aaagcactta ataccacaga cagctcctcc
cctccaagcc tggcagatgg gatagacgcc agtggaggag
gaggcattca tgtcatcagc agagaccagt ccacacccat
catcgaggtc gaaggtcccc tcctctcaga cactcatgtc
acatttaagc tcaccatgcc cagcccaatg ccagagtacc
tcaatgtgca ctacatctgc gagtcagcat cccgcctgct
tttcctctcc atgcactggg ccaggtcaat cccagcgttt
caggcacttg ggcaggactg caacaccagc ctggtgcggg
cctgctggaa tgagctcttc actctgggcc tggcccagtg
tgcccaggtc atgagcctct ctaccatcct ggcggccatt
gtcaaccacc tacagaacag catccaggaa gataaacttt
ctggagaccg gataaagcaa gttatggagc acatctggaa
gcttcaggag ttctgtaaca gtatggccaa gctggatata
gacggctatg agtatgcata ccttaaagct atagttctct
ttagccccga tcatccaggt ttgaccagca caagccagat
tgaaaaattc caagaaaagg cacagatgga attgcaggac
tatgttcaga aaacctactc agaagacacc taccgattgg
cccggattct cgttcgcctg ccggcactca ggctgatgag
ctccaacata acagaagaac ttttttttac tggtctcatt
ggcaatgtgt cgatagacag cataatcccc tacatcctca
agatggagac agcagagtat aacggccaga tcaccggagc
cagtctatag
NM_001192061.1 B. taurus
(SEQ ID NO: 5) (1836 bp)
atgaccagcc cctccccgcg catccagata atctccactg
attctgcggt agcctcacct cagcgcattc agggctctga
gcctgcctct ggctcactga agaaagagaa gatatgtatc
gtgaccgacc agcagacggg gcagaagatc cagatcgtca
ccgcggtgga cgcctccgga tcccccaagc agcagttcat
cctgaccagc cccgatggag ctggaactgg gaaggtgatc
ctggcttccc cggagacctc cagtgccaag cagctcatat
tcaccacctc ggacaacctc gtccccggca ggatccaaat
cgtcacggac tctgcttctg tggagcgttt gctggggaag
accgacgtcc agcggcccca ggtggtggag tactgtgtgg
tgtgcggcga caaagcctca ggccgccact atggggctgt
cagctgcgaa ggctgcaaag gtttcttcaa aaggagcgtg
aggaaaaacc tgacctacag ctgccggagc agccaggact
gcatcatcaa caaacaccac cggaaccgct gccagttctg
ccggctgaag aagtgcctgg agatgggcat gaaaatggag
tctgtgcaga gtgaacggaa accctttgac gtgcagcggg
agaaaccaag caattgtgct gcgtccactg agaagatcta
catccggaag gacctgcgga gtcctctgat agccacgccg
acgttcgtgg ccgagaagga tggggcgaga caaacaggtc
ttcttgatcc agggatgctt gtgaacatcc agcagccttt
gatccgtgag gacggaacgg ttctcctggc cacggactgc
aaggctgaaa caagccaggg agccctgggc acactggcaa
atgtagtcac ttccctcgcc aacctcagtg gaccccttaa
caatggtgac actgcagaaa cgcagccgga ggaccagtct
gcaagtgaga tcactcgggc atttgatacc ttagcaaagg
cacttaacac cacagacagc gcctcgcctc cgagcctggc
ggacgggatc gatggcagcg gcggcggggg catccatgtc
atcagcagag accagtccac gcccatcatt gaggtcgagg
gccccctcct ctcagacact cacgtcacat ttaagctcac
gatgcccagc ccgatgcccg agtacctcaa cgtgcactac
atctgcgagt cggcctcacg cctgctcttc ctctccatgc
actgggccag gtccatcccc gccttccagg ccctcgggca
ggactgcaac accagcctgg tgcgggcctg ctggaacgag
ctcttcaccc tcggcctggc ccagtgcgcc caggtcatga
gcctctccac catcctggct gccatcgtca accacctgca
gaacagcatc caggaagata aactttctgg agaccggata
aagcaagtca tggagcacat ctggaagctc caggagttct
gtaacagtat ggcgagactg gatatagacg gctatgagta
tgcatacctt aaagctatag ttctctttag ccccgatcat
ccaggtttga ccagcacaag ccagattgaa aaattccaag
aaaaggcgca gatggagctg caggactacg ttcagaaaac
ctactcggaa gacacctacc ggttggcccg cattctcgtc
cgcctgccgg cactcaggct gatgagttcc agcataacgg
aagagctgtt ttttactggc ctcattggca acgtttcaat
agacagcatc atcccctaca tcctcaagat ggagacagcg
gagtataacg gccagatcac cggagccagc ctatag
NM_011630.3 M. musculus
(SEQ ID NO: 6) (1791 bp)
atgaccagcc cctccccgcg catccagata atttccaccg
actctgcggt agcctcacct cagcgcattc agattgtaac
agaccagcag acaggacaaa agatccagat agtcaccgca
gtggatgcct ctggatcctc taaacagcag ttcatcctaa
ccagcccaga tggagctgga actgggaagg tgatcctggc
ttctccggaa acatccagtg ccaagcagct catattcacc
acctcggaca accttgtccc tggcaggatc cagatcgtca
cggattctgc ttctgtggag cgtttgctgg ggaaggcaga
cgtccagcgg ccccaggtgg tggagtactg tgtggtctgt
ggcgacaaag cctctggccg gcactatggg gctgtcagtt
gtgaaggttg caaaggtttc ttcaaaagga gcgtgaggaa
gaatctgacc tacagctgtc ggagcagcca agactgcatc
atcaacaagc accaccgtaa ccgctgccag ttctgccggc
tgaagaagtg cctggagatg ggcatgaaaa tggagtctgt
acagagtgaa cggaagccct ttgatgtgca acgggagaaa
ccaagcaatt gtgctgcttc cactgagaag atctatatcc
ggaaagacct gagaagtcct ctgatagcca ctcccacatt
tgtggcagac aaagatggag caagacaaac aggtcttctt
gatccaggga tgcttgtgaa catccaacag cctttgatac
gtgaggatgg tacagttctc ctggccgcgg attccaaggc
tgaaacaagc caaggagctc taggtacact ggcaaatgta
gtgacctctt tggccaacct gagtgaatct ttgaacaacg
gtgatgcttc agaaatgcag ccagaggacc agtctgcaag
tgagattact cgggcatttg acaccttagc gaaagcactt
aataccacag atagtgcttc acctccaagc ctggcagatg
ggatagatgc tagtggagga gggagtatcc atgtcatcag
cagagatcag tcaacaccca tcattgaagt tgaaggccct
ctcctttcag acacacatgt cacattcaag cttacaatgc
ccagtcctat gccagagtac ctcaatgtac attacatctg
tgagtctgca tcccgcctgc ttttcctctc catgcactgg
gcaaggtcaa tcccagcctt ccaggcactt ggacaggact
gtaataccag cctggtgagg gcctgctgga atgagctctt
cactcttggc ctggcccagt gtgcccaggt catgagtctc
tccaccatcc tggcagccat tgtcaaccac ctacagaaca
gcatccagga agataagctt tctggtgacc ggataaagca
agtgatggag cacatctgga agctgcagga gttctgtaac
agcatggcga aactggatat agacggctat gagtacgcat
accttaaagc tatagttctc tttagtcccg atcatccagg
tttgacaggc acaagccaga ttgagaaatt tcaggagaag
gcacagatgg aattacagga ctatgtgcag aaaacctact
ctgaagatac ttacagattg gccaggattc ttgtccgcct
accagcactc aggctcatga gctccaacat aacagaagaa
ctttttttta ctggtctcat tggcaatgtt tcaatagaca
gcataattcc ttacatcctc aagatggaga cagcagaata
taatggccag atcactggag ccagtctata g
NM_017323.1 R. norvegicus
(SEQ ID NO: 7) (1791 bp)
atgaccagcc cctccccgcg catccagata atttccaccg
actctgcggt acgctcacct cagcgcattc agattgtaac
agaccagcag acaggacaaa agctccagat agtcaccgca
gtggacgcct ctggatcctc taaacagcag ttcatcctaa
ccagcccaga tggagctgga actgggaagg tgatcctggc
ttctccagag acatccagtg ccaagcagct catattcacc
acctcagaca accttgtccc tggcaggatc cagatcgtca
cggattctgc ttctgtggag cgattgctgg ggaaggcaga
cgtccagcgg ccccaggtgg tggagtactg tgtggtctgt
ggtgacaaag cctctggccg acactatggg gctgtcagtt
gtgaaggttg caaaggtttc ttcaaaagga gtgtgaggaa
gaatctgacc tatagctgtc gtagcagcca agactgcatc
atcaacaagc accaccgcaa ccgctgccag ttctgccggc
tgaagaagtg cctggagatg ggcatgaaga tggagtctgt
acagagtgaa cggaagccct ttgatgtgca acgggagaaa
ccaagcaatt gtgctgcttc cactgagaag atctatatcc
ggaaagacct gagaagtcct ctgatagcca ctcccacatt
tgtggcagac aaagatggat caagacagac tggtcttctt
gatccaggga tgcttgtgaa catccaacag cctttgatac
gtgaggatgg tacagttctt ttggccacgg attccaaggc
tgaaacaagc caaggagctc taggtacact ggcaaatgta
gtgacctctc tggccaacct gagtgaatct ttgaacaatg
gtgatgcttc agaaatgcag ccagaggacc agtctgcaag
tgaaattaca cgggcatttg acaccctagc gaaagcactt
aataccacag atagtgcttc acctccaagc ctggcagatg
ggatagatgc tagtggagga gggagtatcc atgtcatcag
cagagatcag tcaacaccca tcattgaagt cgaaggccct
ctcctttcag acacacatgt cacatttaag cttacaatgc
ctagtcctat gccagagtac ctcaatgtac attacatctg
cgagtctgca tcccgcctgc ttttcctctc catgcactgg
gcaaggtcaa tcccagcctt ccaggcactt ggacaggact
gcaataccag cctggtacgg gcctgctgga atgagctctt
cactcttggc ctggcccagt gtgcccaggt catgagtctc
tccaccatcc tggcagccat tgtcaaccac ttacagaaca
gcatccagga agataagctt tctggtgacc ggataaagca
agtcatggag cacatctgga agctgcagga gttctgtaac
agcatggcaa aactggatat agacggtcat gagtacgcat
accttaaagc tatagttctc tttagtcccg atcatccagg
tttgacaggc acaagccaga ttgagaaatt tcaggagaag
gcacagatgg aattacaaga ctatgtgcag aaaacctact
cagaagacac atacagattg gccaggattc ttgtccgcct
gccagcactc aggcttatga gctccaatat aacagaagaa
ctttttttta ctggtctcat tggcaatgtt tcaatagaca
gcataattcc ctacatccta aagatggaga cagcagaata
taatggccag atcaccggag ccagtctata g
XM_004944725.2 G. gallus
(SEQ ID NO: 8) (1890 bp)
atggcaacca atatggaggt gctggctcag caggtaatgg
agactcagca ggtggctgag gtacagacta ttcagaattc
actatctgat tctccagtga tgaccagccc ttcccaacgt
atccagatta tttccacaga ttcctctgta ggctcaccac
agcgcattca gattgtgact gatcagcaga caggtcaaaa
aatccagata gtgacagcag tggactcatc tgtgtctcca
aagcaacagt ttattttagc cagtccagat ggaactggtg
caggaaaggt gattttggca gcacctgaga catctaatgc
caaacaactt atctttacaa ccgcagacaa cgttgtgcca
ggcagaattc agatagtgac agactctgct tcggtggaac
gtttgctggg aaaagctgat gttcagcggg cccaagtagt
agaatattgc gtggtctgtg gtgataaagc atcaggtcgt
cactatggtg ctgtcagttg tgagggatgc aaaggtttct
ttaaaaggag tgtgaggaag aatctgacct acagttgtcg
tagcaaccaa gactgtatca tcaataaaca ccatcggaat
cgctgccagt tttgtaggct taagaaatgt ctagagatgg
gcatgaaaat ggaatctgtt caaagtgaaa gaaaaccttt
tgatgtgcag cgtgaaaaac caaccaactg tgcagcttcc
actgaaaaaa tatatatcag aaaagaccta cgaagccctc
taatagcaac tccaacgttt gttgcagata aagatggggc
acggtctgca ggtcttcttg atccaggaat gcttgtgaat
attcaacagc ctttgatcag ggatgatggt accattctcc
tagctgctga ctctaaggct gagacaagcc agggtgcttt
aggaacacta gcaaatgttg taacatctct tgctaacctc
agtgactcac taaataatgg agatacttct gaaattcaac
aagaagagca atccgccagt gagatttcac gggcatttga
tactttggcc aaagctctca gtaccacaga tggtacagca
gttcctaact tggcagatgg gatggatcct acaggaggag
ggaatattca tgtaatcagt agagatcagt caacaccaat
tattgaagtg gaaggaccct tacttacaga tacacatgtt
acatttaagc tgacaatgcc cagtccaatg ccagagtatc
tcaatgtaca ctacatctgt gagtcagcat cacggctact
tttcttatct atgcactggg ctagatccat acctgcattt
caagcacttg gccaggagtg taatacaagc cttgtgcgtg
cctgctggaa tgagctgttt accttaggcc tggcacagtg
tgcacaagtg atgagtctgt caactatcct agcagctatt
gtcaaccatc ttcagaacag catacaggaa gataagcttt
ctggagacag aataaagcaa gtcatggaac acatctggaa
acttcaagag ttctgtaaca gcatggccaa gattgatatc
gatggatatg aatatgcata ccttaaagct atagtcctct
ttagtcccga tcatcctggt ctgaacagtt caacccaaat
agaaaaattc caggagaagg cacagatgga attacaggac
tatgtgcaaa aaacgtatcc agaagatact tataggctag
cccggattct agttcgcctg ccagcactta ggctgatgag
ctctagcatt actgaggaac tattttttac tggtctcatt
ggaaatgttc caattgacag cataatcccc tacattctca
aaatggaaac agcagaatat aatggtcaaa taactggcac
atccgcatag
XM_002938236.4 X. tropicalis
(SEQ ID NO: 9) (1788 bp)
atgacaagct cctctcagcg catccaaatt atctctgcag
actctgccgt gtcatcccca cagcgcatcc agattgtaac
agatcagcag acaggtcaaa agatccagat tgtgacagct
gtagattctt caatatctcc caaacagcaa tttatactga
ccagtcccga tggatctgga acagggaagg tgatactggc
aactcctgaa tccacgagta caaagcaagt tatcttcact
gctgaaaaca ttgtccctgg aagaatacag atagtaacag
atgcagcttc tgttgagcga ctccttggaa agggggatgt
gcagcgccct cagatcatag aatactgtgt tgtttgtggc
gacaaggcat ctggtcgcca ctatggtgca gttagctgtg
aggggtgcaa gggtttcttc aaaagaagtg ttaggaagag
cttgacgtac agttgccgga gcagtcaaga ctgcgtcatc
aataagcacc accgcaatcg atgtcagttc tgtcggctta
aaaaatgcct ggaaatgggc atgaaaatgg aatctgttca
gagtgagaga aaaccttttg atatccagag ggaaaaggca
tcgaactgtg cagcgtcaac agaaaaaatc tacatcagaa
aggacctaag aagtccgctc atagcaactc caacatttgt
ctctgataaa gatggtgcaa ggcaaacagg acttttggac
cctggggtgc tagtgaatat tcagcaacca cttataagag
aagatggcac tgtgattttg tcttcagatg caaagacaga
agcaagtcag ggcggacttg gaacactggc aaatgttgtg
acatcactag caaacctgac agagtctctg aataatgggg
acacagctga tgtccatcca gaggaccaat attcaagtga
aataactcga gcatttgata ccttagcaaa agcacttaat
acaacagagg gctctcctgt acataacctg gcagatggaa
tagatgcttc cactggaggg aacattcaca taattagccg
ggatcaggca actcctatac tggaagttga aggacccctt
ctttcagaca cacatgtgac atttaagctt acaatgccaa
gtccaatgcc agagtatctg aatgtgcact atatctgtga
gtcggcatcc cgcttgcttt tcctctccat gcattgggcc
agatccattc ctgctttcca agctctgggg caggattgta
acacaaacct tgtgcgtgcc tgttggaatg agctttttac
cctgggtctt gcacagtgtt ctcaagtgat gagtctttcc
actatccttg cagccattgt caaccatcta caaaacagca
tacaagaaga caagctttct ggagacagaa taaagcaagt
catggagcat atctggaagc tccaggagtt ctgtaacagt
atgaccaagt tgggtattga tggatatgaa tatgcctacc
ttaaagcagt agtccttttc agcccagatc acccgggtct
ttccagcacg gcacaaatag agaaattcca ggaaaaagcc
cagatggagc tgcaggacta tgttcagaaa acctatcctg
atgatacata taggctagcc cggattttag ttcgacttcc
agctctcaga ctcatgagct ccaacatcac tgaagaactt
ttttttactg gccttatcgg caacgttcct attgacagta
tcatccctta catcctcaag atggagacag cggagtataa
tgggcagatc acgggggccg gtgtatag
NM_001123294. D. rerio
(SEQ ID NO: 10) (1872 bp)
atgacaacca atgtggagct gctggctcag cagatattga
ccactgagca gctttcagag gttcagttgt ctccgtcagg
cagttcagtg atgagcggct ctcctcagcg cattcagatc
atctccaccg agccctcggt caccagccca cagagaatac
agatcgtcac agagcagcag acggggcaga agatccagat
agtgacggct ctggactcct ctgtgcccaa gcagcagttc
ctcctggcct ctcctgatgg ctctccagcg ggtaaagtcc
tgctggcgtc cccagagagc tccagcgcca aacagctcat
cttcgccact gccgacagcc tggtacccgg cagaatacag
tttgtgactg atgccgtgtc cgtggagagg cttctgggta
aaggcacaga cctcagccga gttcagccaa ttgaatactg
tgtggtgtgt ggagataaag cctcagggag gcattatgga
gctgtgagct gtgagggctg taagggtttc ttcaagcgga
gcgtgcgcaa gagtctgacc tacagctgca ggagtaacca
ggactgtgtg atcaacaaac accaccgcaa ccgctgccag
ttctgccggc tccgcaagtg cctggagatg ggcatgaaga
tggagtctgt acaaagtgag cggaaaccca tcgatcttcc
tcgagaaaaa cctgcgaact gtgcagcgtc tacagagaaa
atctacatca ggaaagacct gcgcagtccg cttatcgcaa
cacccacctt catctctgag aaagatagct cacggtccaa
gctgttggat tctgggatgc tcgtcaatat ccagcaaccc
ttaatacagg ctgatggcac attactgtta gcaacagaca
agacggagtc ggggcagggt gatctgggga cactcgctaa
tgtggtcaca tcattagcaa acctgaacga ttcgctcagt
aatggagaag caacagacgg gcagctggag gagtcgccca
gtgaaatcac acgtgccttt gacactctgg ctaaagcgct
gaaccccggc gagctgacgg agagccagag tctgtctgag
gtggacggag tgggcggagc tacaatacag gtgatcagca
gagatcagat ctcgccactc atagaggtgg agggaccgct
gctcaccgac acacatgtca gctttaagct gacgatgccc
agcccgatgc cggagtatct gaatgtacat tatatctgtg
agtcggcttc acgtctcctc ttcctctcca tgcactgggc
gagatctatc ccagcattcc ttgctctcgg gcaggagtgt
aacaccgctc tggttcgggc ctgctggaat gagctgttta
ttctgggtct ggctcagtgt gctcagatca tgagtctgtc
caccatcctc accgccatcg tcaatcatct gcagtccagc
atccaggacg tagacaagct atcaagtgaa cgcatcaaac
tcgtgatgga gcacatctgg aagctgcagg agttctgcaa
cagcatggcg aagctgcaga cggacgctta tgaatacgca
tacctaaaag ccatcgtgct cttcagtcca gatcatccag
gtttgagtag ctgcagccag attgaaaagt ttcaggagaa
agcgcagatg gagctccagg actatgtgca gaagacctat
ccggatgaaa cctacaggtt ggctcgaatc ctgctgcgat
taccggcgtt gcgtttgatg agttccagca taaccgagga
gctgttcttc accggcctca tcggaaacgt accgatcgac
agcatcatcc catatatcct caaaatggag actgcagact
acaacagcca aatcacagcc ccctccgtgt ga

Included in Table 1 are variations of 1, 2, 3, 4, 5, 6, 7, 8, 9, 10, 11, 12, 13, 14, 15, 16, 17, 18, 19, 20, or more nucleotides on the 5′ end, on the 3′ end, or on both the 5′ and 3′ ends, of the nucleic acid sequences.

Included in Table 1 are RNA nucleic acid molecules (e.g., thymines replaced with uredines), nucleic acid molecules encoding orthologs of the encoded proteins, as well as DNA or RNA, nucleic acid molecules comprising, consisting essentially of, or consisting of:

1) a nucleotide sequence having at least 30%, 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of SEQ ID NO: 1-10, or a biologically active or inactive fragment thereof;
2) a nucleotide sequence having at least 30%, 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of SEQ ID NO: 1-10, or a biologically active or inactive fragment thereof, comprising at least one or more (e.g., one, two, three, four, five, six, seven, eight, nine, ten, or more) conserved nucleotides within the DBD, variable N-terminal domain, non-conserved hinge, or LBD of Nr2c2;
3) a nucleotide sequence having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or more nucleic acids, or any range in between, inclusive such as between 200 and 600 nucleotides;
4) a nucleotide sequence having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or more nucleic acids, or any range in between, inclusive such as between 200 and 600 nucleic acids, comprising at least one or more conserved ligand binding nucleotides;
5) a biologically active or inactive fragment of an nucleotide sequence of SEQ ID NO: 1-10 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or more nucleic acids, or any range in between, inclusive such as between 200 and 600 nucleic acids; or
6) a biologically active or inactive fragment of an nucleotide sequence of SEQ ID NO: 1-10 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or more nucleic acids, or any range in between, inclusive such as between 200 and 600 nucleic acids, comprising at least one or more conserved ligand binding nucleotides.

Any of the aforementioned Nr2c2 nucleic acid molecules can have a biological function of the full-length Nr2c2 nucleic acid (e.g., enhance Treg differentiation, maturation, activity, or function), or lack a biological function of the full-length Nr2c2 nucleic acid (e.g., block/reduce Treg differentiation, maturation, activity, or function).

TABLE 2
Nr2c2 (amino acids)

NP_003289.2 Homo sapiens
(SEQ ID NO: 11) (615 aa)
MTSPSPRIQI ISTDSAVASP QRIQGSEPAS GPLSVFTSLN
KEKIVTDQQT GQKIQIVTAV DASGSPKQQF ILTSPDGAGT
GKVILASPET SSAKQLIFTT SDNLVPGRIQ IVTDSASVER
LLGKTDVQRP QVVEYCVVCG DKASGRHYGA VSCEGCKGFF
KRSVRKNLTY SCRSNQDCII NKHHRNRCQF CRLKKCLEMG
MKMESVQSER KPFDVQREKP SNCAASTEKI YIRKDLRSPL
IATPTFVADK DGARQTGLLD PGMLVNIQQP LIREDGTVLL
ATDSKAETSQ GALGTLANVV TSLANLSESL NNGDTSEIQP
EDQSASEITR AFDTLAKALN TTDSSSSPSL ADGIDTSGGG
SIHVISRDQS TPIIEVEGPL LSDTHVTFKL TMPSPMPEYL
NVHYICESAS RLLFLSMHWA RSIPAFQALG QDCNTSLVRA
CWNELFTLGL AQCAQVMSLS TILAAIVNHL QNSIQEDKLS
GDRIKQVMEH IWKLQEFCNS MAKLDIDGYE YAYLKAIVLF
SPDHPGLTST SQIEKFQEKA QMELQDYVQK TYSEDTYRLA
RILVRLPALR LMSSNITEEL FFTGLIGNVS IDSIIPYILK
METAEYNGQI TGASL
AP_001158643.1 P. troglodytes
(SEQ ID NO: 12) (615 aa)
MTSPSPRIQI ISTDSAVASP QRIQGSEPAS GPLSVLTSLN
KEKIVTDQQT GQKIQIVTAV DASGSPKQQF ILTSPDGAGT
GKVILASPET SSAKQLIFTT SDNLVPGRIQ IVTDSASVER
LLGKTDVQRP QVVEYCVVCG DKASGRHYGA VSCEGCKGFF
KRSVRKNLTY SCRSNQDCII NKHHRNRCQF CRLKKCLEMG
MKMESVQSER KPFDVQREKP SNCAASTEKI YIRKDLRSPL
IATPTFVADK DGARQTGLLD PGMLVNIQQP LIREDGTVLL
ATDSKAETSQ GALGTLANVV TSLANLSESL NNGDTSEIQP
EDQSASEITR AFDTLAKALN TTDSSSSPSL ADGIDTSGGG
SIHVISRDQS TPIIEVEGPL LSDTHVTFKL TMPSPMPEYL
NVHYICESAS RLLFLSMHWA RSIPAFQALG QDCNTSLVRA
CWNELFTLGL AQCAQVMSLS TILAAIVNHL QNSIQEDKLS
GDRIKQVMEH IWKLQEFCNS MAKLDIDGYE YAYLKAIVLF
SPDHPGLTST SQIEKFQEKA QMELQDYVQK TYSEDTYRLA
RILVRLPALR LMSSNITEEL FFTGLIGNVS IDSIIPYILK
METAEYNGQI TGASL
AP_002798750.1 M. mulatta
(SEQ ID NO: 13) (648 aa)
MATNMEGLVQ HRVGTQQVAE VTRTQTSRPE SPGMTSPSPR
IQIISTDSAV ASPQRIQGSE PASGPLSVFT SLNKEKIVTD
QQTGQKIQIV TAVDASGSPK QQFILTSPDG AGTGKVILAS
PETSSAKQLI FTTSDNLVPG RIQIVTDSAS VERLLGKTDV
QRPQVVEYCV VCGDKASGRH YGAVSCEGCK GFFKRSVRKN
LTYSCRSNQD CIINKHHRNR CQFCRLKKCL EMGMKMESVQ
SERKPFDVQR EKPSNCAAST EKIYIRKDLR SPLIATPTFV
ADKDGARQTG LLDPGMLVNI QQPLIREDGT VLLATDSKAE
TSQGALGTLA NVVTSLANLS ESLNNGDTSE IQPEDQSASE
ITRAFDTLAK ALNTTDSSSS PSLADGIDTS GGGSIHVISR
DQSTPIIEVE GPLLSDTHVT FKLTMPSPMP EYLNVHYICE
SASRLLFLSM HWARSIPAFQ ALGQDCNTSL VRACWNELFT
LGLAQCAQVM SLSTILAAIV NHLQNSIQED KLSGDRIKQV
MEHIWKLQEF CNSMAKLDID GYEYAYLKAI VLFXSDHPGL
TSTSQIEKFQ EKAQMELQDY VQKTYSEDTY RLARILVRLP
ALRLMSSNIT EELFFTGLIG NVSIDSIIPY ILKMETAEYN
GQITGASL
AP_005632175.1 C. lupus
(SEQ ID NO: 14) (629 aa)
MATNMEGLVQ HRVGTQQVAE VPRTQTSRPE SPGMTSPSPR
IQIISTDSAV ASPQRIQIVT DQQTGQKIQI VTAVDASGSP
KQQFILTSPD GAGTGKVILA SPETSSAKQL IFTTSDNLVP
GRIQIVTDSA SVERLLGKAD VQRPQVVEYC VVCGDKASGR
HYGAVSCEGC KGFFKRSVRK NLTYSCRSNQ DCIINKHHRN
RCQFCRLKKC LEMGMKMESV QSERKPFDVQ REKPSNCAAS
TEKIYIRKDL RSPLIATPTF VADKDGARQT GLLDPGMLVN
IQQPLIREDG TVLLATDSKA ETSQGALGTL ANVVTSLANL
SESLNNGDAS EMQPEDQSAS EITRAFDTLA KALNTTDSSS
PPSLADGIDA SGGGGIHVIS RDQSTPIIEV EGPLLSDTHV
TFKLTMPSPM PEYLNVHYIC ESASRLLFLS MHWARSIPAF
QALGQDCNTS LVRACWNELF TLGLAQCAQV MSLSTILAAI
VNHLQNSIQE DKLSGDRIKQ VMEHIWKLQE FCNSMAKLDI
DGYEYAYLKA IVLFSPDHPG LTSTSQIEKF QEKAQMELQD
YVQKTYSEDT YRLARILVRL PALRLMSSNI TEELFFTGLI
GNVSIDSIIP YILKMETAEY NGQITGASL
NP_001178990.1 B. taurus
(SEQ ID NO: 15) (611 aa)
MTSPSPRIQI ISTDSAVASP QRIQGSEPAS GSLKKEKICI
VTDQQTGQKI QIVTAVDASG SPKQQFILTS PDGAGTGKVI
LASPETSSAK QLIFTTSDNL VPGRIQIVTD SASVERLLGK
TDVQRPQVVE YCVVCGDKAS GRHYGAVSCE GCKGFFKRSV
RKNLTYSCRS SQDCIINKHH RNRCQFCRLK KCLEMGMKME
SVQSERKPFD VQREKPSNCA ASTEKIYIRK DLRSPLIATP
TFVAEKDGAR QTGLLDPGML VNIQQPLIRE DGTVLLATDC
KAETSQGALG TLANVVTSLA NLSGPLNNGD TAETQPEDQS
ASEITRAFDT LAKALNTTDS ASPPSLADGI DGSGGGGIHV
ISRDQSTPII EVEGPLLSDT HVTFKLTMPS PMPEYLNVHY
ICESASRLLF LSMHWARSIP AFQALGQDCN TSLVRACWNE
LFTLGLAQCA QVMSLSTILA AIVNHLQNSI QEDKLSGDRI
KQVMEHIWKL QEFCNSMARL DIDGYEYAYL KAIVLFSPDH
PGLTSTSQIE KFQEKAQMEL QDYVQKTYSE DTYRLARILV
RLPALRLMSS SITEELFFTG LIGNVSIDSI IPYILKMETA
EYNGQITGAS L
NP_035760.1 M. musculus
(SEQ ID NO: 16) (596 aa)
MTSPSPRIQI ISTDSAVASP QRIQIVTDQQ TGQKIQIVTA
VDASGSSKQQ FILTSPDGAG TGKVILASPE TSSAKQLIFT
TSDNLVPGRI QIVTDSASVE RLLGKADVQR PQVVEYCVVC
GDKASGRHYG AVSCEGCKGF FKRSVRKNLT YSCRSSQDCI
INKHHRNRCQ FCRLKKCLEM GMKMESVQSE RKPFDVQREK
PSNCAASTEK IYIRKDLRSP LIATPTFVAD KDGARQTGLL
DPGMLVNIQQ PLIREDGTVL LAADSKAETS QGALGTLANV
VTSLANLSES LNNGDASEMQ PEDQSASEIT RAFDTLAKAL
NTTDSASPPS LADGIDASGG GSIHVISRDQ STPIIEVEGP
LLSDTHVTFK LTMPSPMPEY LNVHYICESA SRLLFLSMHW
ARSIPAFQAL GQDCNTSLVR ACWNELFTLG LAQCAQVMSL
STILAAIVNH LQNSIQEDKL SGDRIKQVME HIWKLQEFCN
SMAKLDIDGY EYAYLKAIVL FSPDHPGLTG TSQIEKFQEK
AQMELQDYVQ KTYSEDTYRL ARILVRLPAL RLMSSNITEE
LFFTGLIGNV SIDSIIPYIL KMETAEYNGQ ITGASL
NP_059019.1 R. norvegicus
(SEQ ID NO: 17) (596 aa)
MTSPSPRIQI ISTDSAVRSP QRIQIVTDQQ TGQKLQIVTA
VDASGSSKQQ FILTSPDGAG TGKVILASPE TSSAKQLIFT
TSDNLVPGRI QIVTDSASVE RLLGKADVQR PQVVEYCVVC
GDKASGRHYG AVSCEGCKGF FKRSVRKNLT YSCRSSQDCI
INKHHRNRCQ FCRLKKCLEM GMKMESVQSE RKPFDVQREK
PSNCAASTEK IYIRKDLRSP LIATPTFVAD KDGSRQTGLL
DPGMLVNIQQ PLIREDGTVL LATDSKAETS QGALGTLANV
VTSLANLSES LNNGDASEMQ PEDQSASEIT RAFDTLAKAL
NTTDSASPPS LADGIDASGG GSIHVISRDQ STPIIEVEGP
LLSDTHVTFK LTMPSPMPEY LNVHYICESA SRLLFLSMHW
ARSIPAFQAL GQDCNTSLVR ACWNELFTLG LAQCAQVMSL
STILAAIVNH LQNSIQEDKL SGDRIKQVME HIWKLQEFCN
SMAKLDIDGH EYAYLKAIVL FSPDHPGLTG TSQIEKFQEK
AQMELQDYVQ KTYSEDTYRL ARILVRLPAL RLMSSNITEE
LFFTGLIGNV SIDSIIPYIL KMETAEYNGQ ITGASL
AP_004944782.1 G. gallus
(SEQ ID NO: 18) (629 aa)
MATNMEVLAQ QVMETQQVAE VQTIQNSLSD SPVMTSPSQR
IQIISTDSSV GSPQRIQIVT DQQTGQKIQI VTAVDSSVSP
KQQFILASPD GTGAGKVILA APETSNAKQL IFTTADNVVP
GRIQIVTDSA SVERLLGKAD VQRAQVVEYC VVCGDKASGR
HYGAVSCEGC KGFFKRSVRK NLTYSCRSNQ DCIINKHHRN
RCQFCRLKKC LEMGMKMESV QSERKPFDVQ REKPTNCAAS
TEKIYIRKDL RSPLIATPTF VADKDGARSA GLLDPGMLVN
IQQPLIRDDG TILLAADSKA ETSQGALGTL ANVVTSLANL
SDSLNNGDTS EIQQEEQSAS EISRAFDTLA KALSTTDGTA
VPNLADGMDP TGGGNIHVIS RDQSTPIIEV EGPLLTDTHV
TFKLTMPSPM PEYLNVHYIC ESASRLLFLS MHWARSIPAF
QALGQECNTS LVRACWNELF TLGLAQCAQV MSLSTILAAI
VNHLQNSIQE DKLSGDRIKQ VMEHIWKLQE FCNSMAKIDI
DGYEYAYLKA IVLFSPDHPG LNSSTQIEKF QEKAQMELQD
YVQKTYPEDT YRLARILVRL PALRLMSSSI TEELFFTGLI
GNVPIDSIIP YILKMETAEY NGQITGTSA
AP_002938282.2 X. tropicalis
(SEQ ID NO: 19) (595 aa)
MTSSSQRIQI ISADSAVSSP QRIQIVTDQQ TGQKIQIVTA
VDSSISPKQQ FILTSPDGSG TGKVILATPE STSTKQVIFT
AENIVPGRIQ IVTDAASVER LLGKGDVQRP QIIEYCVVCG
DKASGRHYGA VSCEGCKGFF KRSVRKSLTY SCRSSQDCVI
NKHHRNRCQF CRLKKCLEMG MKMESVQSER KPFDIQREKA
SNCAASTEKI YIRKDLRSPL IATPTFVSDK DGARQTGLLD
PGVLVNIQQP LIREDGTVIL SSDAKTEASQ GGLGTLANVV
TSLANLTESL NNGDTADVHP EDQYSSEITR AFDTLAKALN
TTEGSPVHNL ADGIDASTGG NIHIISRDQA TPILEVEGPL
LSDTHVTFKL TMPSPMPEYL NVHYICESAS RLLFLSMHWA
RSIPAFQALG QDCNTNLVRA CWNELFTLGL AQCSQVMSLS
TILAAIVNHL QNSIQEDKLS GDRIKQVMEH IWKLQEFCNS
MTKLGIDGYE YAYLKAVVLF SPDHPGLSST AQIEKFQEKA
QMELQDYVQK TYPDDTYRLA RILVRLPALR LMSSNITEEL
FFTGLIGNVP IDSIIPYILK METAEYNGQI TGAGV
NP_001116766.1 D. rerio
(SEQ ID NO: 20) (623 aa)
MTTNVELLAQ QILTTEQLSE VQLSPSGSSV MSGSPQRIQI
ISTEPSVTSP QRIQIVTEQQ TGQKIQIVTA LDSSVPKQQF
LLASPDGSPA GKVLLASPES SSAKQLIFAT ADSLVPGRIQ
FVTDAVSVER LLGKGTDLSR VQPIEYCVVC GDKASGRHYG
AVSCEGCKGF FKRSVRKSLT YSCRSNQDCV INKHHRNRCQ
FCRLRKCLEM GMKMESVQSE RKPIDLPREK PANCAASTEK
IYIRKDLRSP LIATPTFISE KDSSRSKLLD SGMLVNIQQP
LIQADGTLLL ATDKTESGQG DLGTLANVVT SLANLNDSLS
NGEATDGQLE ESPSEITRAF DTLAKALNPG ELTESQSLSE
VDGVGGATIQ VISRDQISPL IEVEGPLLTD THVSFKLTMP
SPMPEYLNVH YICESASRLL FLSMHWARSI PAFLALGQEC
NTALVRACWN ELFILGLAQC AQIMSLSTIL TAIVNHLQSS
IQDVDKLSSE RIKLVMEHIW KLQEFCNSMA KLQTDAYEYA
YLKAIVLFSP DHPGLSSCSQ IEKFQEKAQM ELQDYVQKTY
PDETYRLARI LLRLPALRLM SSSITEELFF TGLIGNVPID
SIIPYILKME TADYNSQITA PSV

Included in Table 2 are variations of 1, 2, 3, 4, 5, 6, 7, 8, 9, 10, 11, 12, 13, 14, 15, 16, 17, 18, 19, 20, or more amino acids on the 5′ end, on the 3′ end, or on both the 5′ and 3′ ends, of the amino acid sequences.

Included in Table 2 are orthologs of the proteins, as well as polypeptide molecules comprising, consisting essentially of, or consisting of:

1) an amino acid sequence having at least 30%, 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of SEQ ID NO: 11-20, or a biologically active or inactive fragment thereof;
2) an amino acid sequence having at least 30%, 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of SEQ ID NO: 11-20, or a biologically active fragment thereof, comprising at least one or more (e.g., one, two, three, four, five, six, seven, eight, nine, ten or more) conserved residues within the DBD, variable N-terminal domain, non-conserved hinge, or LBD of Nr2c2;
3) an amino acid sequence having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, or more amino acids, or any range in between, inclusive such as between 200 and 400 amino acids;
4) an amino acid sequence having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, or more amino acids, or any range in between, inclusive such as between 200 and 400 amino acids, comprising at least one or more conserved ligand binding residues;
5) a biologically active or inactive fragment of an amino acid sequence of SEQ ID NO: 11-20 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, or more amino acids, or any range in between, inclusive such as between 200 and 6400 amino acids; or
6) a biologically active fragment of an amino acid sequence of SEQ ID NO: 11-20 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, or more amino acids, or any range in between, inclusive such as between 200 and 400 amino acids, comprising at least one or more conserved ligand binding residues.

Any of the aforementioned Nr2c2 polypeptides can have a function of the full-length

Nr2c2 polypeptide (e.g., enhance Treg differentiation, maturation, activity, or function), or lack a function of the full-length Nr2c2 polypeptide (e.g., block/reduce Treg differentiation, maturation, activity, or function).

As used herein, “RNA interference (RNAi)” is an evolutionally conserved process whereby the expression or introduction of RNA of a sequence that is identical or highly similar to a target nucleic acid (e.g., Nr2c2) results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target nucleic acid. This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target nucleic acids.

“Short interfering RNA” (siRNA), also referred to herein as “small interfering RNA” is defined as an agent which functions to inhibit expression of an Nr2c2 nucleic acid, e.g., by RNAi. An siRNA may be chemically synthesized, may be produced by in vitro transcription, or may be produced within a host cell. In some embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. The siRNA is capable of promoting RNA interference through degradation or specific post-transcriptional gene silencing (PTGS) of the target messenger RNA (mRNA).

In some embodiments, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (g., Stewart, et al. (2003) RNA 9(4):493-501).

As used herein, the term “substantially decreased” and grammatical equivalents thereof refer to a level, amount, concentration of a parameter, such as a chemical compound, a metabolite, a nucleic acid, a polypeptide, or a physical parameter (pH, temperature, viscosity, etc.), measured in a sample that has a decrease of at least 10%, preferably about 20%, more preferable about 40%, even more preferable about 50% and still more preferably a decrease of more than 75% when compared to the level, amount, or concentration of the same chemical compound, nucleic acid, polypeptide, physical parameter, or microorganism in a control sample. In some embodiments, the parameter is not detectable in a subject sample, while it is detectable in a control sample.

As used herein, the term “substantially increased” and grammatical equivalents thereof refer to a level, amount, concentration of a parameter, such as a chemical compound, a metabolite, a nucleic acid, a polypeptide, a or physical parameter (pH, temperature, viscosity, etc.), measured in a sample that has an increase of at least 30%, preferably about 50%, more preferable about 75%, and still more preferably an increase of more than 100% when compared to the level, amount, or concentration of the same chemical compound, nucleic acid, polypeptide, physical parameter, or microorganism in a control sample. In some embodiments, the parameter is detectable in a subject sample, while it is not detectable in a control sample.

As used herein, the terms “treat,” “treating,” and “treatment” include: (1) preventing a pathological condition, disorder, or disease, i.e. causing the clinical symptoms of the pathological condition, disorder, or disease not to develop in a subject that may be predisposed to the pathological condition, disorder, or disease but does not yet experience any symptoms of the pathological condition, disorder, or disease; (2) inhibiting the pathological condition, disorder, or disease, i.e. arresting or reducing the development of the pathological condition, disorder, or disease or its clinical symptoms; or (3) relieving the pathological condition, disorder, or disease, i.e. causing regression of the pathological condition, disorder, or disease or its clinical symptoms. These terms encompass also prophylaxis, therapy and cure. Treatment means any manner in which the symptoms of a pathological condition, disorder, or disease are ameliorated or otherwise beneficially altered. Preferably, the subject in need of such treatment is a mammal, more preferable a human.

As used herein, “Tregs” or Regulatory T cells have pluripotent anti-inflammatory effects on multiple cell types. In particular, they control the activation of innate and adaptive immune cells. Tregs acting in an antigen-specific manner reduce effector T cell activation and function, for example, after effector T cells have successfully mounted an attack against an invading pathogen, or to suppress reactivity to self-antigen and thereby prevent autoimmune disease.

Two subsets of Tregs are classified according to the location at which they develop in vivo. Naturally-occurring Tregs (nTreg) develop in the thymus and suppress self-reactive immune responses in the periphery, whereas adaptive Tregs (aTreg) develop in the periphery from conventional CD4⁺ T cells to ensure tolerance to harmless antigens, including those derived from, for example, food and intestinal flora.

Both subsets of Treg cells are characterized by expression of high levels of CD25 and the transcription factor Foxp3. Tregs are thought to inhibit the antigen-specific expansion and/or activation of self-reactive effector T cells and to secrete suppressive cytokines, including TGF or IL-10. Because of their potential to provide antigen-specific immune regulation without generalized immunosuppression, Tregs have been contemplated for use in cell-based therapy for inflammatory or autoimmune disorders.

As used herein, a “variant” may comprise a “biologically active fragment” or a “biologically inactive fragment” of a polypeptide, and refers to a polypeptide (e.g., Nr2c2) having the amino acid sequence of the polypeptide in which is altered one or more amino acid residues (e.g., any of the polypeptide sequence set forth in Table 2). The variant may have “conservative” changes, wherein a substituted amino acid has similar structural or chemical properties (e.g., replacement of leucine with isoleucine). A variant may have “nonconservative” changes (e.g., replacement of glycine with tryptophan). Analogous variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “variant,” when used in the context of a polynucleotide sequence (e.g., any of the Nr2c2 nucleotide sequences set forth in Table 1), may encompass a polynucleotide sequence related to that of a particular gene or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variantion is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

### 2. Compositions, Agents, and the Like, for Manipulating Nuclear Receptor Level, Expression, Function, or Activity to Affect Treg Differentiation, Maturation, Activity, or Function

In certain embodiments, the invention relates to compositions that can modulate the activity of, level of, or expression of a target nuclear receptor. In one embodiment, the target nuclear receptor is Nr2c2. In some embodiments, such modulation may include compositions that deplete, suppress, reduce, or decrease Nr2c2 to block or reduce Treg maturation, differentiation, activity, or function. In some embodiments, such modulation may include compositions that activate, initiate, increase, or stimulate Nr2c2 to enhance Treg maturation, differentiation, activity, or function.

In some embodiments, the depletion, suppression, reduction, or decrease of Nr2c2 to block or reduce Treg maturation, differentiation, activity, or function may be mediated using gene editing techniques and methods known in the art, including but not limited to, siNA, Clustered Regularly Interspaced Short Palindromic Repeats-Caspase 9 (CRISPR/Cas9), Transcription activator-like effector nucleases (TALEN), or zinc-finger nuclease (ZFN).

CRISPR together with cas (CRISPR-associated) genes comprise an adaptive immune system that provides acquired resistance against invading foreign nucleic acids in bacteria and archaea (Barrangou et al. (2007) Science 315:1709-12). CRISPR consists of arrays of short conserved repeat sequences interspaced by unique variable DNA sequences of similar size called spacers, which often originate from phage or plasmid DNA (Barrangou et al. (2007) Science 315:1709-12; Bolotin et al. (2005) Microbiology 151:2551-61; Mojica et al. (2005) J. Mol. Evol, 60:174-82). The CRISPR-Cas system functions by acquiring short pieces of foreign DNA (spacers) which are inserted into the CRISPR region and provide immunity against subsequent exposures to phages and plasmids that carry matching sequences (Barrangou et al. (2007) Science 315:1709-12; Brouns et al. (2008) Science 321:960-64). It is this CRISPR-Cas interference/immunity that enables crRNA-mediated silencing of foreign nucleic acids (Horvath & Barrangou (2010) Science 327:167-70; Deveau et al. (2010) Annu. Rev. Microbiol. 64:475-93; Marraffini & Sontheimer (2010) Nat. Rev. Genet. 11:181-90; Bhaya et al. (2011) Annu. Rev. Genet. 45:273-97; Wiedenheft et al. (2012) Nature 482:331-338).

Use of CRISPR constructs that rely upon the nuclease activity of the Cas9 protein (Makarova et al. (2011) Nat. Rev. Microbiol. 9:467-77) coupled with a synthetic guide RNA (gRNA) has recently revolutionized genomic-engineering, allowing for unprecedented manipulation of DNA sequences. CRISPR/Cas9 constructs are simple and fast to synthesize and can be multiplexed. Cleavage by the CRISPR system requires complementary base pairing of the gRNA to a 20-nucleotide DNA sequence and the requisite protospacer-adjacent motif (PAM), a short nucleotide motif found 3′ to the target site (Jinek et al. (2012) Science 337: 816-821). One can, theoretically, target any unique gene sequence in the genome using CRISPR technology. The DNA binding specificity of the PAM sequence, which varies depending upon the species of origin of the specific Cas9 employed, provides one constraint. Currently, the least restrictive and most commonly used Cas9 protein is from S. pyogenes, which recognizes the sequence NGG, and thus, any unique 21-nucleotide sequence in the genome followed by two guanosine nucleotides (N₂₀NGG) can be targeted.

Additional genome-editing technologies may be used in any of the methods described herein, including zinc fingers nucleases (ZFN) (Porteus, and Baltimore (2003) Science 300: 763; Miller et al. (2007) Nat. Biotechnol. 25:778-785; Sander et al. (2011) Nature Methods 8:67-69; Wood et al. (2011) Science 333:307) and transcription activator-like effectors nucleases (TALEN) (Wood et al. (2011) Science 333:307; Boch et al. (2009) Science 326:1509-1512; Moscou and Bogdanove (2009) Science 326;1501; Christian et al. (2010) Genetics 186:757-761; Miller et al. (2011) Nat. Biotechnol. 29:143-148; Zhang et al. (2011) Nat. Biotechnol. 29:149-153; Reyon et al. (2012) Nat. Biotechnol. 30:460-465). ZFN and TALEN have the ability to generate targeted genome modifications with precision.

TALEN leverages artificial restriction enzymes generated by fusing a TAL effector DNA-binding domain to a DNA cleavage domain. Transcription activator-like effectors (TALEs) can be quickly engineered to bind practically any desired DNA sequence. By combining such an engineered TALE with a DNA cleavage domain (which cuts DNA strands), one can engineer restriction enzymes that will specifically cut any desired DNA sequence (Boch J (2011) Nature Biotechnology 29 (2):135-6). When these restriction enzymes are introduced into cells, they can be used for gene editing or for genome editing in situ, a technique known as genome editing with engineered nucleases.

ZFNs are a class of engineered DNA-binding proteins that facilitate targeted editing of the genome by creating double-strand breaks in DNA at user-specified locations. Each ZFN consists of two functional domains. One domain is a DNA-binding domain comprised of a chain of two-finger modules, each recognizing a unique hexamer (6 bp) sequence of DNA. Two-finger modules are stitched together to form a Zinc Finger Protein, each with specificity of ≥24 bp. The second domain is a DNA-cleaving domain comprised of the nuclease domain of Fok I. When the DNA-binding and DNA-cleaving domains are fused together, a highly-specific pair of ‘genomic scissors’ are created. Double-strand breaks are important for site-specific mutagenesis in that they stimulate the cell's natural DNA-repair processes, namely homologous recombination and Non-Homologous End Joining (NHEJ). By implementing established, field proven methods, these processes are harnessed to generate precisely targeted genomic edits, resulting in cell lines with targeted gene deletions, integrations, or modifications. Both ZFN and TALEN pairs require synthesizing large and unique recognition proteins for a given DNA target site.

Other methods for the depletion, suppression, reduction, or decrease of Nr2c2 to block or reduce Treg maturation, differentiation, activity, or function may be mediated by using RNAi targeted to Nr2c2. The term “short interfering nucleic acid”, “siNA”, “siRNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating expression of the Nr2c2 gene (e.g., any of the nucleotide sequences set forth in Table 1), for example by mediating RNA interference “RNAi” or gene silencing in a sequence-specific manner (Bass (2001) Nature 411:428-429; Elbashir et al. (2001) Nature, 411:494-498; and Kreutzer et al. International PCT Publication No. WO 00/44895; Zernicka-Goetz et al. International PCT Publication No. WO 01/36646; Fire, International PCT Publication No. WO 99/32619; Plaetinck et al. International PCT Publication No. WO 00/01846; Mello and Fire International PCT Publication No. WO 01/29058; Deschamps-Depaillette International PCT Publication No. WO 99/07409; and Li et al. International PCT Publication No. WO 00/44914; Allshire (2002) Science 297:1818-1819; Volpe et al. (2002) Science 297:1833-1837; Jenuwein (2002) Science 297:2215-2218; and Hall et al. (2002) Science 297:2232-2237; Hutvagner and Zamore (2002) Science 297:2056-60; McManus et al. (2002) RNA 8:842-850; Reinhart et al. (2002) Gene & Dev., 16:1616-1626; and Reinhart & Bartel (2002) Science, 297:1831).

For example, the siNA can be a double-stranded polynucleotide molecule comprising self-complementary sense and antisense regions, wherein the antisense region comprises nucleotide sequence that is complementary to nucleotide sequence in any of the Nr2c2 nucleic acid molecule or a portion thereof, and the sense region having nucleotide sequence corresponding to the Nr2c2 nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in an Nr2c2 nucleic acid molecule or a portion thereof and the sense strand comprises nucleotide sequence corresponding to the Ne2c2 nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s).

In certain embodiments, the siNA can be a circular single-stranded polynucleotide having two or more loop structures and a stem comprising self-complementary sense and antisense regions, wherein the antisense region comprises nucleotide sequence that is complementary to nucleotide sequence in an Nr2c2 nucleic acid molecule or a portion thereof and the sense region having nucleotide sequence corresponding to the Nr2c2 nucleic acid sequence or a portion thereof, and wherein the circular polynucleotide can be processed either in vivo or in vitro to generate an active siNA molecule capable of mediating RNAi.

In certain embodiments, the siNA molecule comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siNA molecules of the invention comprise nucleotide sequence that is complementary to nucleotide sequence of an Nr2c2 gene. In another embodiment, the siNA molecule of the invention interacts with nucleotide sequence of an Nr2c2 gene in a manner that causes inhibition of expression of the Nr2c2 gene.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

The term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, the term RNAi is meant to be equivalent to other terms used to describe sequence specific RNA interference, such as post transcriptional gene silencing, translational inhibition, or epigenetics. For example, siNA molecules of the invention can be used to epigenetically silence genes at both the post-transcriptional level or the pre-transcriptional level. Epigenetic regulation of gene expression by siNA molecules of the invention can result from siNA mediated modification of chromatin structure to alter gene expression (see, for example, Allshire (2002) Science 297:1818-1819; Volpe et al. (2002) Science 297:1833-1837; Jenuwein (2002) Science 297:2215-2218; and Hall et al. (2002) Science 297:2232-2237).

In some embodiments, the depletion, suppression, reduction, or decrease of Nr2c2 to block or reduce Treg maturation, differentiation, activity, or function may be mediated by using an Nr2c2 variant, which is a biologically inactive Nr2c2 fragment or mutant. Such a biologically-inactive Nr2c2 fragment or mutant may comprise any of the nucleotide or amino acid sequences set forth in Table 1 or Table 2.

In some embodiments, the depletion, suppression, reduction, or decrease of Nr2c2 to block or reduce Treg maturation, differentiation, activity, or function may be mediated by using a ligand or chemical compound that may antagonize Nr2c2 function, level, expression, or activity, and thus, reduce/block Treg maturation, differentiation, activity, or function. Such ligands would be favorable to reduce Treg inhibition of anti-tumor responses, and as an anti-cancer or anti-tumor therapy. In some embodiments, it may be beneficial to reduce Treg activity in the context of some infections, to relieve the brakes on anti-microbial immune responses. Examples, of anti-microbial immune responses may be directed to, but not limited to, any viral, bacterial, and/or fungal infections as set forth above in section 1.

Another embodiment of the invention relates to modulating the activity of, level of, or expression of Nr2c2. Such modulation may include the activation, intiation, increase, or stimulation of Nr2c2 function, level, expression, or activity to enhance Treg differentiation, function, maturation, or activity. In some embodiments, the activation, intiation, increase, or stimulation of Nr2c2 may be mediated using a ligand or chemical compound that is an agonist, or acts as an agonist of Nr2c2. In some embodiments, Nr2c2 agonists may enhance Treg pools, or Treg maturation, differentiation, activity, or funcation. Such compositions may limit autoimmune, inflammatory diseases, among others. For example, autoimmune diseases include, but are not limited to, rheumatoid arthritis, myasthenia gravis, multiple sclerosis, psoriasis, systemic lupus erythematosus, autoimmune thyroiditis (Hashimoto's thyroiditis), Graves' disease, inflammatory bowel disease, autoimmune uveoretinitis, polymyositis, and certain types of diabetes. A disorder, disease, condition, or illness associated with inflammation, or inflammatory disorder, includes, but are not limited to, septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacteria and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, and pulmonary inflammation.

In some embodiments, ligands or agonists of Nr2c2 may include, but not limited to, polyunsaturated fatty acids (PUFAs), such as omega-3 and -6 fatty acids, and their metabolites such as 15-hydroxyeico-satetraonic acid (15-HETE) and 13-hydroxy octa-deca dieonic acid (13-HODE) and thiazolidinedione (TZD)-rosiglitazone (Xie, S. et al. (2009) Proc Natl Acad Sci USA 106, 13353-13358). In some embodiments, ligands or agonists of Nr2c2 may include, but not limited to, retinoids, including all-trans-retinoic acid, retinol (Zhou, X. E. et al. (2011) J Biol Chem 286, 2877-2885). In some embodiments, ligands or agonists of Nr2c2 may include, but not limited to, keto-mycolic acid from Mycobacterium tuberculosis cell wall lipids (Dkhar, H. K. et al. (2014) J. Immunol. 193, 295-305). In some embodiments, ligands or agonists of Nr2c2 may include, but not limited to, γ-linoleic acid has also been reported that can activate Nr2c2 and its target gene (Tsai, N. P. et al. (2009) Biochimica Biophysica Acta 1789, 734-740).

In some embodiments, the activation, intiation, increase, or stimulation of Nr2c2 to enhance Treg differentiation, function, maturation, or activity may be mediated by using an Nr2c2 variant, which is a biologically active Nr2c2 fragment or mutant. Such a biologically active Nr2c2 fragment or mutant may comprise any of the nucleotide or amino acid sequences set forth in Table 1 or Table 2.

In some embodiments, assays used to identify agents (e.g., agonists or antagonists of Nr2c2) useful in the methods of the present invention include a reaction between a polypeptide comprising a sequence selected from SEQ ID NO: 11-20, or a fragment thereof, and one or more assay components. The other components may be either a test compound (e.g. the potential agent), or a combination of test compounds and an Nr2c2 protein or fragment thereof. In some embodiments, assays used to identify agents useful in the methods of the present invention include a reaction between a nucleic acid comprising a sequence selected from SEQ ID NO: 1-10, or a fragment thereof, and one or more assay components. The other components may be either a test compound (e.g. the potential agent), or a combination of test compounds and an Nr2c2 nucleic acid or fragment thereof. Agents identified via such assays, may be useful, for example, for preventing or treating cancer, among others; or, limiting autoimmune or inflammatory diseases, among others, as set forth above. In some embodiments, it may be beneficial to reduce Treg activity in the context of some infections, to relieve the brakes on anti-microbial immune responses. Examples, of anti-microbial immune responses may be directed to, but not limited to, any viral, bacterial, and/or fungal infections as set forth above in section 1.

Agents useful in the methods of the present invention may be obtained from any available source, including systematic libraries of natural and/or synthetic compounds. Agents may also be obtained by any of the numerous approaches in combinatorial library methods known in the art, including: biological libraries, small molecule libraries, chemical libraries, peptoid libraries (libraries of molecules having the functionalities of peptides, but with a novel, non-peptide backbone which are resistant to enzymatic degradation but which nevertheless remain bioactive) (Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other approaches are applicable to peptide, non-peptide oligomer, or small molecule libraries of compounds (Lam (1997) Anticancer Drug Des. 12:). Agents useful in the methods of the present invention may be identified, for example, using assays for screening candidate or test compounds which deplete Nr2c2 to block or reduce Treg maturation, or increase Nr2c2 to enhance Treg pools or Treg activity.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91: 11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233). Libraries of agents may be presented in solution (e.g., Houghten (1992) Biotechniques 13:412-421), or on beads (Lam (1991) Nature 354:82-84), chips (Fodor (1993) Nature 364:555-556), bacteria and/or spores, (Ladner et al. U.S. Pat. No. 5,223,409), plasmids (Cull et al,. (1992) Proc Natl Acad Sci USA 89:1865-1869) or on phage (Scott and Smith (1990) Science 249:386-390; Devlin (1990) Science 249:404-406; Cwirla et al (1990) Proc. Natl. Acad. Sci. 87:6378-6382; Felici (1991) J. Mol. Biol. 222:301-310; Ladner, supra).

The present invention further pertains to agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. For agents determined as possessing high efficacy with low toxicity and side effects, it is within the scope of this invention to further formulate said agent as a pharmaceutical composition as described herein. It is also within the scope of this invention to further use an agent, or pharmaceutical compositon comprising same, in any of the therapeutic methods described herein.

Pharmaceutical Compositions

Another embodiment of the present invention relates to a pharmaceutical composition, containing at least one agent that modulates the level of, activity of, or expression of an Nr2c2, with a pharmaceutically acceptable carrier. Another embodiment of the present invention relates to a pharmaceutical composition, containing at least one agent that depletes, knocks down, reduces, or suppresses the level of, activity of, or expression of an Nr2c2, with a pharmaceutically acceptable carrier. Another embodiment of the present invention relates to a pharmaceutical composition, containing at least one agent that activates, initiates, increases, or stimulates the level of, activity of, or expression of an Nr2c2, with a pharmaceutically acceptable carrier. Another embodiment of the present invention relates to an Nr2c2 variant, or biologically active fragment thereof, with a pharmaceutically acceptable carrier. Another embodiment of the present invention relates to an Nr2c2 variant, or biologically inactive fragment thereof, with a pharmaceutically acceptable carrier. Another embodiment of the present invention relates to an Nr2c2 agonist, with a pharmaceutically acceptable carrier. Another embodiment of the present invention relates to an Nr2c2 antagonist, that is with a pharmaceutically acceptable carrier. In some embodiments, the composition includes a combination of multiple (e.g., two or more) agents of the invention.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; or (3) in a drink form, or sachet, that is mixed prior to ingestion.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art. Pharmaceutical compositions can be administered to a subject according to methods known in the art. For example, nucleic acids encoding a protein or an antisense molecule can be administered to a subject as described above, e.g., using a viral vector. Cells can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Pharmaceutical compositions of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof. The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween™ 60, Span™ 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods.

Formulations suitable for vaginal administration may be presented as pessaries, tampons, creams, gels, pastes, foams or spray formulations containing in addition to the active ingredient such carriers as are known in the art to be appropriate. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Pharmaceutical compositions of the invention are used to provide controlled release pharmaceutical formulations containing as active ingredient one or more agents of the invention (“controlled release formulations”) in which the release of the active ingredient are controlled and regulated to allow less frequency dosing or to improve the pharmacokinetic or toxicity profile of a given active ingredient.

Effective dose of active ingredient depends at least on the nature of the condition being treated, toxicity, whether the pharmaceutical composition is being used prophylactically (lower doses) or against an active disease, the method of delivery, and the pharmaceutical formulation, and will be determined by the clinician using conventional dose escalation studies. It can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

One or more pharmaceutical compositions may be administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient.

### 3. Screening Assays and Methods.

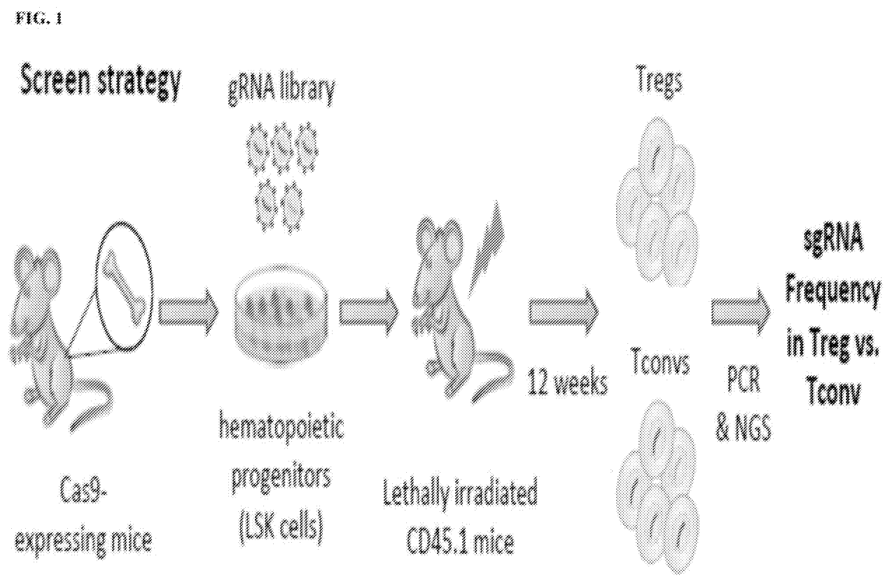

Another aspect of the present invention relates to screening assays. In some embodiments, the screening assays include using a specific pooled-CRIPSR/Cas9 in vivo screening method. In other embodiments, siRNA, TALEN, ZFN, and any other gene editing technologies known in the art, may be adapted to the screening assays described herein. In some embodiments, the screening assay is depicted in FIG. 1. Such assays provide a method for identifying if a gene that is specifically required for the differentiation or homeostatic maintenance of a given cell-type relative to others. For example, the sgRNAs that inactivate the particular gene will be under-represented in that cell relative to others. If a gene is generally necessary for differentiation of all cell-types, the representation of the corresponding gRNAs will be decreased everywhere, relative to the starting stem cell pool.

CRISPR/Cas9 system has been applied to knockout specific genes in primary murine and human hematopoietic stem/progenitor cells (HSPCs), and also human peripheral blood mononuclear cell (PBMC)-derived primary T lymphocytes in vitro. The ribonucleoprotein (RNP) particles generated by Cas9 protein pre-complexed with sgRNA were delivered by electroporation (Gundry, M. C. et al. (2016) Cell Rep. 17:1453-1461). The analysis of cell fate was performed in vitro.

Likewise, CRIPSR genome editing was used to correct β-globin (HBB) gene mutation in human in human hematopoietic stem cells (HSC) in vitro. The function of those HSCs and erythrocytes differentiation were measured by in vivo engraftment into immunodeficient non-obese diabetic (NOD)-severe combined immunodeficiency (SCID) Il2rg−/− (NSG) mice. Cas9 protein complexed with sgRNA were electroporated into HSCs, and donor homologous recombination vectors were delivered by AAV6 (Dever, D. P. et al. (2016) Nature 539:384-389; Bak, R. et al. (2018) Nat. Protoc. 13:358-376). CRISPR/Cas9 genetic editing system was also used to making ablation of the CCRS gene in HSCs. Efficient CCRS ablation was achieved in vivo in long-term reconstituted NOD/Prkdcscid/IL-2Rγnull mice which confers HIV-1 resistance in vivo (Xu, L. et al. (2017) Mol. Ther. 25, 1782-1789).

Mouse models of myeloid malignancy with combinatorial genetic lesions were generated using CRISPR-Cas9 genome editing. Single or multiple sgRNAs were transduced into mouse Lineage−/Scal+/cKit+ (LSK) cells. LSK cells were then transplanted lethally irradiated recipient mice, caused significant myeloid skewing of hematopoiesis with reduction of B cells and leukocytosis in some mice (Heckl, D. et al. (2014) Nat. Biotechnol. 32:941-946). A similar experimental acute myeloid leukemia model was also developed by others. A specific gene was targeted to validate its function (Tzelepis, K. et al. (2016) Cell Rep. 17, 1193-1205).

The present screening methods provide advantages over the aforementioned in vitro or ex vivo screening methods where CRIPSR/Cas9 system has been applied to knockout genes (Cong, L. et al. (2013) Science 339,819-823; Shalem, O. et al. (2014) Science 343, 84-87; Wang, T. et al. (2014) Science 343, 80-84; Platt, R. J. et al. (2014) Cell 159,440-455; Parnas, O. et al. (2015) Cell 162(3):675-86; Chu, V. T. et al. (2016) Proc Natl Acad Sci USA 113:12514-12519). For example, the screening assays provided herein are performed in vivo using specific pooled-CRIPSR/Cas9. Said pooled CRISPR/Cas9 may be applied to mutagenize any gene of interest (e.g., nuclear receptor) in vivo and characterize the gene's roles in any immune cell (e.g., T cell) differentiation and function. In certain embodiments, the gene of interest is Nr2c2. In certain embodiments, the immune cell is a Treg.

One aspect of the invention relates to an in vivo cell-based assay for screening for targets that modulate a biological response in a cell. Such methods comprise the step of (a) isolating cells (e.g., bone marrow stem cells) from an organism (e.g., transgenic mice); (b) transducing the isolated cells with a vector (e.g., lentiviral), wherein said vector encodes at least one RNA (e.g., single guide RNA (sgRNA)) directed to at least one gene (e.g., nuclear receptor, Nr2c2, among others); (c) reconstituting the cells in legthally irradiated hosts; (d) allowing colonization and differentiation of the cells (e.g., mature immunocytes); (e) characterizing the cells (e.g., flow-cytometry); (f) preparing genomic DNA from the cells; and (g) detecting the levels of gRNA, wherein a decreased level of said gRNA among all the cells relative to the starting cell is indicative that said gene modulates a biological response (e.g., differentiation or homeostatic maintenance, among others) in a cell.

In certain embodiments, the cells includes, but is not limited to, primary cultures of cells, embryonic stem cells, adult stem cells, pluripotent cells, blood cells, germ cells, germ cell precursors, or tissues or organs cells, and any progeny thereof can be used. In certain embodiments, the vector is a viral vector. In some embodiments, the viral vector includes, but is not limited to, retroviruses, adenoviruses, adeno-associated viruses, alphaviruses, and herpes simplex virus. In some embodiments, the vector encodes at least two, three, four, five, six, seven, eight, nine, ten, fifteen, twenty, twenty-five, thirty, thirty-five, forty, forty-five, fifty, or more gRNAs. In some embodiments, the genomic DNAs are barcoded and the sequence of each sgRNA is amplified and sequenced using high throughput sequencing methods known in the art.

### 4. Therapeutic Methods and Uses

Disclosed herein are methods of preventing or treating diseases, conditions, or illnesses that would benefit from reduced or blocked Treg maturation, differentiation, activity, or function. Another embodiment of the present invention relates to methods for treating diseases, conditions, or illnesses that would benefit from enhanced Treg pools, or Treg maturation, differentiation, activity, or function. In certain embodiments, the agent and/or Nr2c2 variant, or fragment thereof, or nucleic acid encoding same, described herein is administered to a subject (e.g., a subject in need thereof). In some embodiments, the agents deplete Nr2c2 function, activity, level, or expression. In some embodiments, the agents increase Nr2c2 function, activity, level, or expression. In some embodiments, Nr2c2 depletion may result in block of Treg maturation. In some embodiments, Nr2c2 increase may result in enhanced Treg pools. In some embodiments, Nr2c2 increase may result in activation, enhancement, stimulation of Treg maturation, differentiation, activity, or function. In certain embodiments, the Nr2c2 variant, or fragment thereof, or nucleic acid encoding same, may comprise biologically active variants. In certain embodiments, the Nr2c2 variant, or fragment thereof, or nucleic acid encoding same, may comprise biologically inactive variants. In such embodiments, the agent and/or Nr2c2 variant, or fragment thereof, or nucleic acid encoding same is contacted to the cell either in vitro or in vivo.

One aspect of the present invention provides therapeutic methods of recovering from, preventing, or treating cancer. Cancer includes, but are not limited to, solid tumors (such as those of the bladder, bowel, brain, breast, endometrium, heart, kidney, lung, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

Additional non-limiting examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, Wilms tumor.

Another aspect of the present invention provides therapeutic methods of treating or preventing a disorder, disease, condition, or illness associated with inflammation, or inflammatory disorder. A disorder, disease, condition, or illness associated with inflammation, or inflammatory disorder, includes, but are not limited to, septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacteria and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, and pulmonary inflammation.

Another aspect of the invention relates to methods useful for modulating an inflammatory response in a subject.

In some embodiments, the methods may involve decreasing the activity, expression, or level of Nr2c2 (e.g., any of the nucleic acid or amino acid Nr2c2 molecules set forth in Table 1 and/or Table 2). Such depletion may be mediated by introducing siNA, CRISPR/Cas9, TALEN, ZFN, or any available gene editing techniques that target Nr2c2 (e.g., any of the nucleic acid or amino acid Nr2c2 molecules set forth in Table 1 and/or Table 2). Nr2c2 depletion may block or reduce Treg maturation. Such a block or reduction in Treg maturation may be useful in situations like cancer, where it would be favorable to reduce Treg inhibition of anti-tumor responses. Additional ways to deplete Nr2c2 may comprise using antagonists of Nr2c2, or introducing into Nr2c2 biological inactive variants of any of the nucleic acid or amino acid Nr2c2 molecules set forth in Table 1 and/or Table 2).

In some embodiments, the methods may involve increasing the level of Nr2c2 protein by introducing into a cell a nucleic acid encoding the Nr2c2 protein (e.g., any of the nucleic acid or amino acid Nr2c2 molecules set forth in Table 1 and/or Table 2) operably linked to a transcriptional regulatory sequence directing the expression of the protein in the cell. Methods for expressing nucleic acids in cells and appropriate transcriptional regulatory elements for doing so are well known in the art. Alternatively, an Nr2c2 biologically active protein, or variant thereof, can be introduced into a cell, usually in the presence of a vector facilitating the entry of the protein into the cells, e.g., liposomes. Nr2c2 proteins can also be linked to transcytosis peptides for that purpose. Additional ways to increase or enhance levels of, activities of, or functions of Nr2c2 may comprise addition of Nr2c2 agonist that might enhance Treg pools or their activity, and thus limit autoimmune or inflammatory diseases, among others.

It will be apparent to a person of skill in the art that a full length Nr2c2 protein or nucleic acid encoding such or a portion thereof (e.g., any of the nucleic acid or amino acid Nr2c2 molecules set forth in Table 1 and/or Table 2) can be used according to the methods described herein. A portion of an Nr2c2 protein may be a biologically active or inactive portion thereof. Portions that are biologically active can be identified according to methods known in the art and using an assay that can monitor the activity of the particular protein. For example, an Nr2c2 protein that is a biologically active portion thereof may be monitor if it enhances Treg pools or their activity. Similarly, an Nr2c2 protein that is a biologically inactive portion thereof may be monitor if it blocks or reduces Treg maturation.

In addition to portions of Nr2c2 proteins, other variants, such as proteins containing a deletion, insertion, or addition of one or more amino acids can be used. Amino acid changes can include one or more conservative amino acid substitutions. Nr2c2 protein, or a biologically active portion thereof, may include one or more conservative amino acid substitutions within the DBD, variable N-terminal domain, or LBD of Nr2c2. Amino acid changes can include one or more nonconservative amino acid substitutions. Nr2c2 protein, or a biologically inactive portion thereof, may include one or more nonconservative amino acid substitutions within the DBD, variable N-terminal domain, or LBD of Nr2c2. Other changes may include one or more conservation or nonsubstitutions for non-naturally occurring amino acids. Additional changes may comprise deletion of any of the one or more conserved regions of Nr2c2 (e.g., deletion of DBD, variable N-terminal domain, or LBD). Additional modifications may comprise pluralities of Nr2c2 conserved domains (e.g., DBD, variable N-terminal domain, or LBD) operable linked to form a fusion protein or polypeptide.

Nr2c2 variants may have at least about 50%, 70%, 80%, 90%, preferably at least about 95%, even more preferably at least about 98% and most preferably at least 99% homology or identity with a wild-type Nr2c2 protein or a domain thereof, e.g., any of the conserved domains. Other Nr2c2 proteins may be encoded by a nucleic acid that is at least about 90%, preferably at least about 95%, even more preferably at least about 98% and most preferably at least 99% homology or identity with a wild-type Nr2c2, e.g., those described herein.

In other embodiments Nr2c2 proteins are fusion proteins, e.g., proteins fused to a transcytosis peptide. Fusion proteins may also comprise a heterologous peptide that can be used to purify the protein and/or to detect it. In other embodiments, non-naturally occurring protein variants are used. Such variants can be peptidomimetics.

Any means for the introduction of Nr2c2 nucleic acid or polypeptide molecules into mammals, human or non-human, or cells thereof may be adapted to the practice of this invention for the delivery of the various nucleic acid or amino acid Nr2c2 molecules set forth in Table 1 and/or Table 2, or vectors and/constructs comprising same, into the intended recipient. In some embodiments, any of nucleic acid or amino acid Nr2c2 molecules set forth in Table 1 and/or Table 2 may be introduced as a “naked” molecule. In some embodiments, the DNA vectors and/constructs comprising any of nucleic acid or amino acid Nr2c2 molecules set forth in Table 1 and/or Table 2 are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. An example of a colloidal system is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences Felgner, et al., (1995) Ann NY Acad Sci 126-139, 1995). Formulation of DNA, e.g. with various lipid or liposome materials, may then be effected using known methods and materials and delivered to the recipient mammal. See, e.g., Canonico et al (1994) Am J Respir Cell Mol Biol 10:24-29; Tsan et al, Am J Physiol 268; Alton et al. (1993) Nat Genet. 5:135-142, and U.S. Pat. No. 5,679,647 by Carson et al.

The expression of any of the amino acid Nr2c2 molecules set forth in Table 2 may be measured, in cells of a subject to whom, e.g., a nucleic acid encoding the protein was administered, can be determined, e.g., by obtaining a sample of the cells of the patient and determining the level of the protein in the sample, relative to a control sample.

In another embodiment, an Nr2c2 protein or biologically active or inactive variant thereof (e.g., any of the amino acid Nr2c2 molecules set forth in Table 2) is administered to the subject such that it reaches the target cells, and traverses the cellular membrane. Nr2c2 polypeptides can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Robert K Scopes Protein Purification: Principles and Practice Third Ed. Springer-Verlag, N.Y. 1994). In one embodiment, the polypeptide is produced as a fusion polypeptide comprising an epitope tag consisting of about six consecutive histidine residues. The fusion polypeptide can then be purified on a N⁺⁺ column. By inserting a protease site between the tag and the polypeptide, the tag can be removed after purification of the peptide on the N⁺⁺ column. These methods are well known in the art and commercial vectors and affinity matrices are commercially available.

Administration of Nr2c2 polypeptides (e.g., any of the amino acid Nr2c2 molecules set forth in Table 2) can be done by mixing them with liposomes, as described above. The surface of the liposomes can be modified by adding molecules that will target the liposome to the desired physiological location.

In some embodiments, an Nr2c2 protein is modified so that its rate of traversing the cellular membrane is increased. For example, the polypeptide can be fused to a second peptide which promotes “transcytosis,” e.g., uptake of the peptide by cells. In one embodiment, the peptide is a portion of the HIV transactivator (TAT) protein, such as the fragment corresponding to residues 37-62 or 48-60 of TAT, portions which are rapidly taken up by cell in vitro (Green and Loewenstein, (1989) Cell 55:1179-1188). In another embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example Derossi et al. (1996) J Biol Chem 271:18188-18193; Derossi et al. (1994) J Biol Chem 269:10444-10450; and Perez et al. (1992) J Cell Sci 102:717-722.

Another aspect of the invention provides a method for treating or preventing a disorder associated with inflammation. In some embodiments, the introduction, treatment, or addition of an Nr2c2 agonist may enhance Treg pools or their activity to limit autoimme or inflammatory diseases, among others. In other embodiments, the inflammatory response is depressed or suppressed. Another aspect of the invention provides a method for treating or preventing cancer. In some embodiments, the introduction, treatment, or addition of an Nr2c2 antagonist may provide an anti-tumor or anti-cancer effect. In other embodiments, the anti-cancer effect is enhanced. A subject may self-administer the pharmaceutical agents (e.g., Nr2c2 agonist or

Nr2c2 antagonist), or any of the pharmaceutical compositions described herein, as desired, or a physician may administer the agents or pharmaceutical compositions. Additionally, a physician or other health care worker may select a delivery schedule. In some embodiments, the pharmaceutical agents (e.g., Nr2c2 agonist or Nr2c2 antagonist) are administered on a routine schedule. A routine refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve, for example, administration of the pharmaceutical agents (e.g., Nr2c2 agonist or Nr2c2 antagonist) on a daily basis for the first week, followed by a monthly basis for several months, and then every three months after that. Any particular combination would be covered by the routine schedule as long as it is determined ahead of time that the appropriate schedule involves administration on a certain day. For use in therapy, an effective amount of the pharmaceutical agents (e.g., Nr2c2 agonist or Nr2c2 antagonist) can be administered to a subject by any mode (see above description in section 2.)

Administering a pharmaceutical composition of any of the Nr2c2 variants, or fragments thereof, or a nucleic acid encoding same (e.g., any of the nucleic acid or amino acid Nr2c2 molecules set forth in Table 1 Table 2) may be delivered by any suitable route of administration, including orally, nasally, as by, for example, a spray, rectally, intravaginally, parenterally, intracisternally and topically, as by powders, ointments or drops, including buccally and sublingually. In certain embodiments the pharmaceutical compositions are delivered generally (e.g., via oral or parenteral administration) (see above description in section 2.)

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds of the invention employed in the pharmaceutical composition at levels lower than that required in order to achieve the desired therapeutic effect and gradually increase the dosage until the desired effect is achieved.

In some embodiments, the methods further comprise administering to the subject an effective amount of an agent that enhances Treg differentiation, function, activity, or maturation in the subject. Examples of such agents (e.g., agonists) include polyunsaturated fatty acids (PUFAs), omega-3 fatty acids, omega-6 fatty acids, 15-HETE, 13-HODE, TZD-rosiglitazone, retinoids, all-trans-retinoic acid, retinol, and keto-mycolic acid. In some embodiments, such an agent is administered at a dose of between 0.5-5 grams per day. In some embodiments, agent is orally administered in doses of between 250 mg-5 grams per day.

Any of the Nr2c2 variant, or fragment thereof, or nucleic acid encoding same (e.g., any of the nucleic acid or amino acid Nr2c2 molecules set forth in Table 1 Table 2) and/or agent can be administered simultaneously (e.g., as separate pharmaceutical compositions/formulations), as a combination (e.g., as a single pharmaceutical composition/formulation), or sequentially (e.g., as separate pharmaceutical compositions/formulations one after the other).

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

As can be appreciated from the disclosure above, the present invention has a wide variety of applications. The invention is further illustrated by the following examples, which are only illustrative and are not intended to limit the definition and scope of the invention in any way.

### Examples

I. Materials and Methods
Mice. CRISPR/Cas9 knockin mice constitutively express CRISPR associated protein 9 (Cst-Cas9) endonuclease (also known Rosa26-Cas9-knockin) were purchased from the Jackson Laboratory. And Cas9 knockin mice were crossed with Foxp3-Thy1.1 reporter mice on C57BL/6 background.
Lentiviral library: Optimized sgRNAs were designed to target the 49 NR genes (4 sgRNAs for each target gene), while limiting off-target effect according to Doench, J.G. et al. (2016, Nature Biotechnology 34, 184-191). Synthesized oligos (including 10 non-targeting control sgRNAs and 4 sgRNAs targeting Foxp3) were annealed and phosphorylated individually and then pooled cloning into the BsmBI digested lentiviral vector (pLKO.3G backbone) between the hU6 promoter and gRNA scaffold (Shalem, O. et al. (2014) Science 343, 84-87). The lentiviral vector also contains a second cassette expressing mRFP or EGFP under the hPGK promoter. To produce lentivirus, 3.8 million 293T cells were seed into a 10 cm dish the day before transfection in DMEM supplemented with 10% fetal bovine serum. For transfection of 293T cells, 9 ug lentiviral plasmids together with 9 ug psPAX2 and 1 ug pVSVG (Addgene) were transfected into cells using TransIT transfection kit (Minis Bio). After 18 hrs, the media was changed to 15 ml DMEM supplemented with 10% fetal bovine serum and 1% BSA. 30 hrs after medium changes, supernatant was collected and filtered through a 0.45 um low protein binding membrane (Millipore). The virues were concentrated using PEG-it virus precipitation solution according to the manufacturer (System Biosciences).
Bone marrow chimera mice reconstitution. Bone marrow hematopoietic stem and progenitor cells (lineage negative, Scal positive, c-kit positive cells, LSKs) were sorted from Cst-Cas9 x Foxp3-Thy1.1 mice by flow cytometry sorting using Astrios MoFlo. LSK cells were then cultured in vitro and infected at an approximate MOI of 10 (lentiviruses titrated on 293T; about 40% LSK cells were infected which indicated that only one copy of sgRNA was introduced into each LSK cell in most cases) with the NR sgRNA lentiviral library on the second day. On the third day, 50,000 LSK cells were transferred intraveneously into lethally irradiated CD45.1 congenic mice.
Analysis of bone marrow chimeras. 10-12 weeks post LSK cell transfer, donor-derived cells in spleen and lymph nodes were surface staining with fluorophore conjugated antibodies. Tregs and Tconvs cells were analyzed or sorted by flow cytometry. For CRISPR pooled screen experiments, genomic DNA were prepared from Tregs and Tconvs sorted from BMC mice. The abundance of sgRNA in those cells were determined by PCR amplification of sgRNA and Illumina barcodes and sequencing primers were added during PCR (as described in Shalem, O. et al. (2014) Science 343, 84-87). PCR apmlicons were gel extracted, quantified, mixed and sequenced using a MiSeq (Illumina).

II. In Vivo CRISPR Pooled Screen Identifies the Novel Role of a Nuclear Receptor in Treg Activation and Maturation, Via Metabolic Control

A modified CRISPR-based library screen was established (Wang, T. et al. (2014) Science 343:80-84) which aimed to assess the importance of each of the 49 NRs that exist in mice in the differentiation and homeostasis of immunologic cell-types (FIG. 1). Congenically-marked bone marrow stem cells from a Cas9-expressing transgenic mouse were transduced with a library of lentiviruses encoding single-guide RNAs (sgRNAs) that targeted each of the 49 NR genes (4 guides per gene, plus 14 control guides, 210 sgRNAs altogether). These cells were then used to reconstitute lethally irradiated hosts. After 10-12 weeks to allow for colonization by the stem cells and differentiation of mature immunocytes, different cell-types were sorted by flow cytometry, their genomic DNA was prepared. From this DNA, the barcodes corresponding to each cell sample and the sequence of each sgRNA were amplified and sequenced in bulk by high-throughput sequencing. The logic of the experiment was that if a gene was specifically required for the differentiation or homeostatic maintenance of a given cell-type relative to others, sgRNAs that inactivate it would be under-represented in that cell relative to others. If a gene was generally necessary for differentiation of all cell-types, the representation of the corresponding gRNAs would be decreased everywhere, relative to the starting stem cell pool.

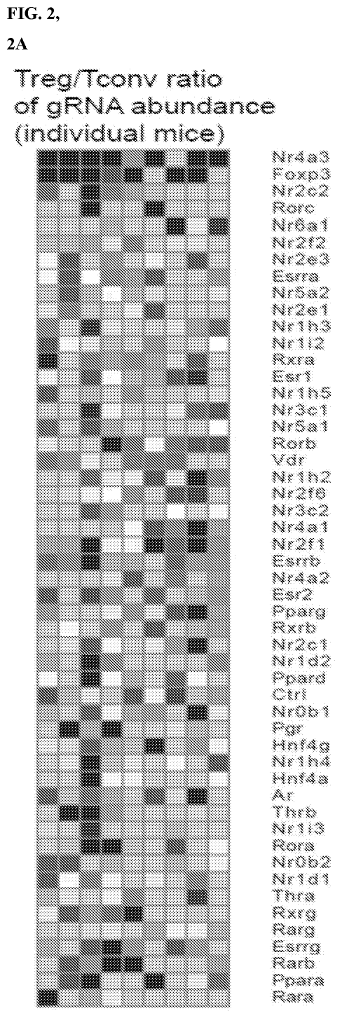

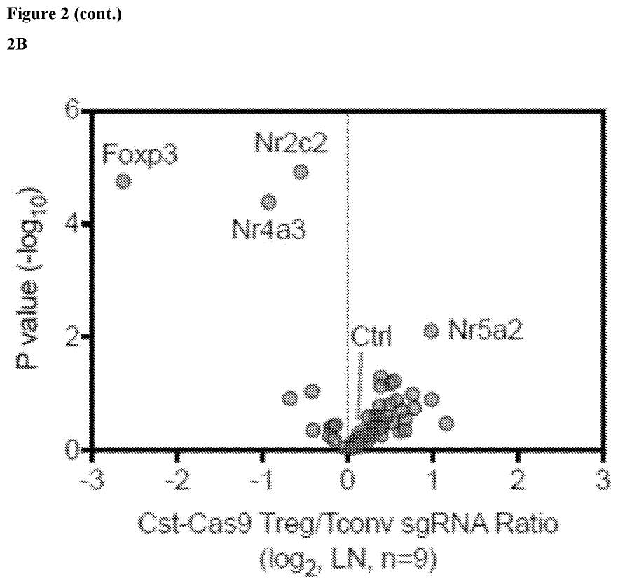

In a first series of experiments with this system, CD4⁺ T cells were focused on, and specifically FoxP3+ T regulatory cells (Treg) (Josefowicz, S.Z. et al. (2012) Annu. Rev. Immunol. 30:531-564) and conventional (Tconv) cells. FIG. 2A displays the relative frequency of sgRNA barcodes in Treg cells from reconstituted mice relative to Tconv cells, taken as a close comparator, (all of the 4 gRNAs for each gene were pooled). These experiments brought forth a small set of genes with biased representation in all informative mice (there is some variation from mouse to mouse in the outcome of these experiments, particularly since there is limited seeding of the thymus by committed progenitors, and not every guide is represented in every mouse). Among these genes showing biased guide representation was (i) FoxP3, the transcription essential for specification of the Treg lineage, which was included as a positive control even though it is not an NR; (ii)Nr4a3 whose importance in Treg homeostasis has been reported by (Fassett, M. S. et al. (2012) Proc Natl Acad Sci USA. 109:3891-3896; Sekiya, T. et al. (2013) Nat. Immunol. 14:230-237); and (iii) Nr2c2 (a.k.a. TR4 (Lee, Y. F. et al. (2002) J Steroid Biochem. Mol Biol. 81:291-308; Lin, S. J. et al. (2017) Curr. Top. Dev. Biol. 125:357-373) whose relationship to Treg physiology had not been recognized previously. The statistical significance of these observations was confirmed by a one-sample t-test represented in FIG. 2B. Note that gRNAs targeting other NRs (Rorc and Nr6a1) were also under-represented in Treg cells of the mice in which they were present, but these were not numerous enough to yield statistical significance in the Treg/Tconv comparison.

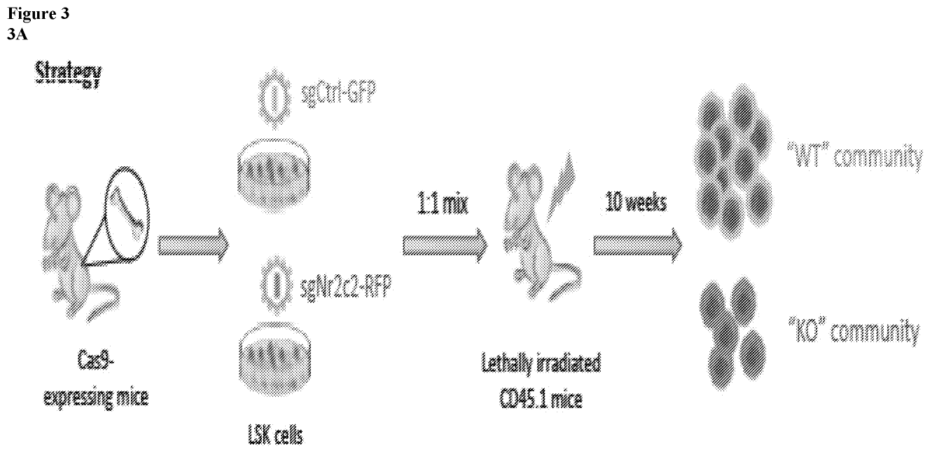

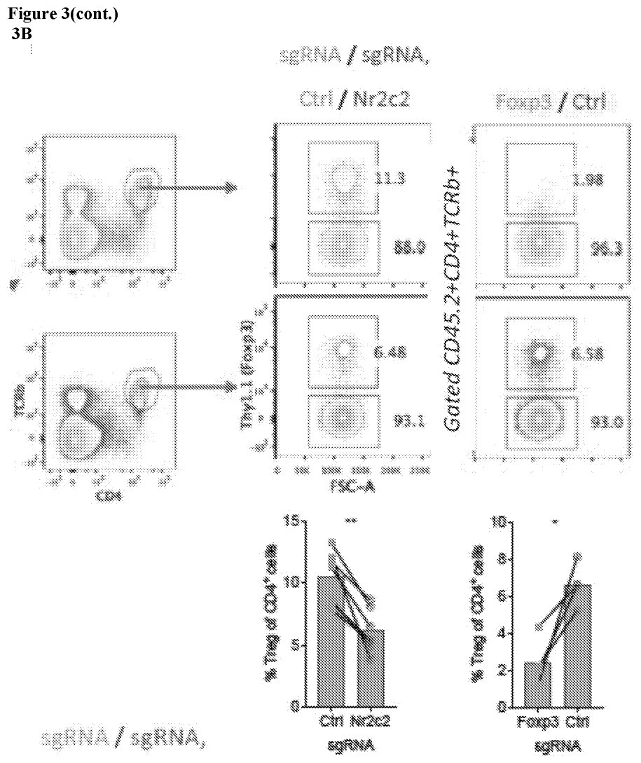

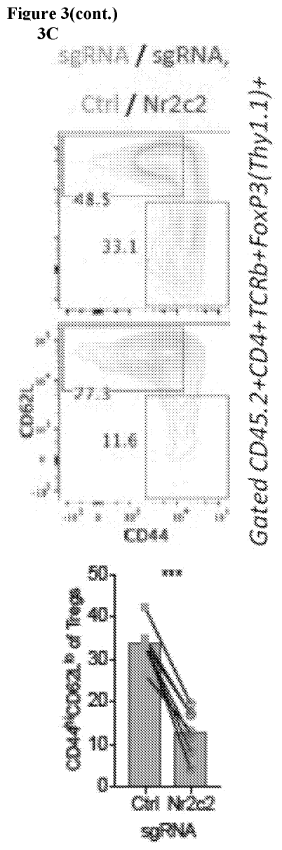

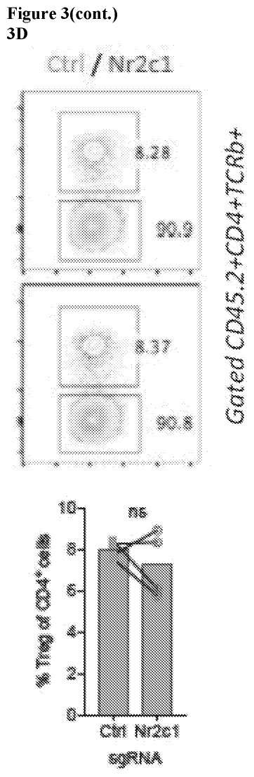

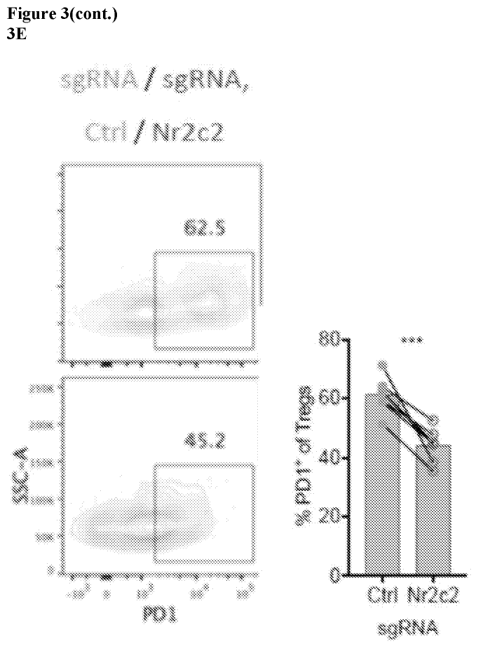

Follow-up experiments were performed to confirm this “hit” by reconstituting irradiated C57B1/6 mice with stem cells transduced with two color-coded lentiviral vectors expressing either an Nr2c2-inactivating sgRNA or a control sgRNA (colon-coded with fluorescent GFP on RFP reporters) allowing a direct comparison of mutant and control cells differentiating in the same environment (FIG. 3A; the stem cells originated from a transgenic mouse carrying several traits: Cas9 for sgRNA-directed genome editing, FoxP3-Thy1.1 to identify Treg cells, CD45.2 congenic interval to determine donor-derived mature cells after transfer). While other parameters such as the proportion of total CD4+ T cells were not affected, the proportion of Treg cells among CD4+ T cells was specifically reduced in the progeny of stem cells in which Nr2c2 was targeted (FIG. 3B); this reduction in Treg cells (41.7% on average) was not as complete as when the Treg determining factor FoxP3 was targeted in similarly reconstituted mice (63.4% on average; FIG. 3B, right). Phenotypic analysis of the residual Nr2c2-deficient Treg shows that they are severely hampered in the ability to enter in the “activated Treg” (aTreg) state characterized by the CD44^{hi}CD62L^{lo} marker combination (FIG. 3C). This block of Treg maturation in the absence of Nr2c2 was not observed with mutation of the close homolog Nr2c1, indicating the specificity of Nr2c2′s function (FIG. 3D). Nr2c2-deficient Treg cells were also defective in the expression of the co-inhibitory molecule PD1 (FIG. 3E), which is required for full Treg activity (Francisco, L. M. et al. (2010) Immunol Rev. 236:219-242), and the target of highly effective checkpoint-inhibition immunotherapy (Topalian, S. L. et al. (2012) N. Engl. J Med. 366:2443-2454). These effects of Nr2c2 on Treg maturation have important functional implications, as aTregs are the most effective at suppressing immune responses and inflammation (Miyara, M. et al. (2009) Immunity 30:899-911; Kleinewietfeld, M. et al. (2005) Blood 105:2877-2886; Smigiel, K. S. et al. (2014) J Exp. Med. 211:121-136; Cretney, E. et al. (2013) Trends Immunol. 34:74-80).

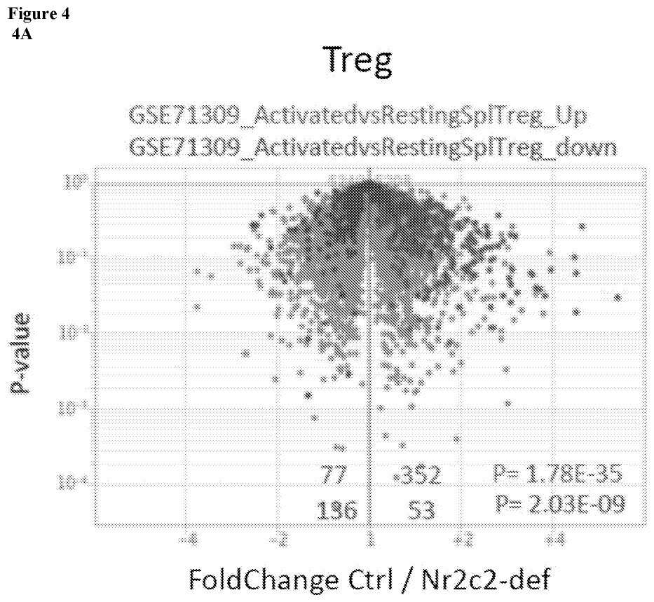

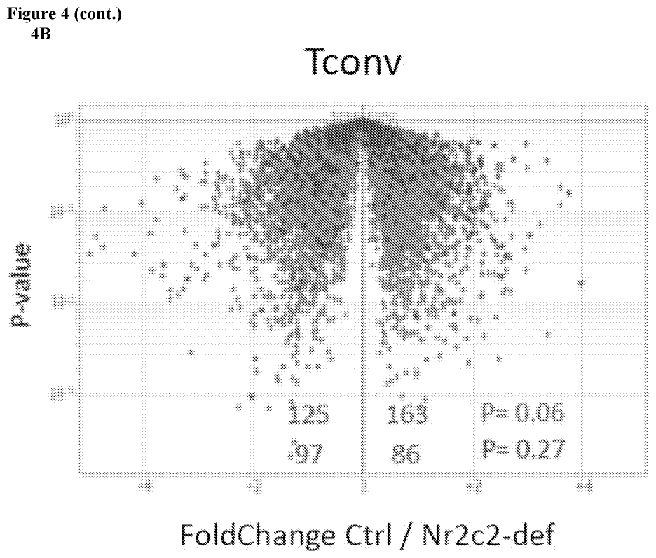

To determine the molecular underpinning of this block, the transcriptomes of wild-type (WT) and Nr2c2 mutant Treg cells (again generated by genome editing in stem cells were compared by RNAseq, followed by reconstitution and differentiation in vivo). Nr2c2 deficiency affected the expression of a sizeable number of transcripts (136 at a FoldChange of 2). Signature analysis revealed a profound shift in the gene expression signature of activated Treg cells (FIG. 4B), confirming the flow cytometry results above, and extending them by revealing a shift in the entire aTreg signature, not limited to a few surface markers. Importantly, these effects were restricted to Treg cells, as the Nr2c2 deficiency did not have the same transcriptional consequences in Tconv cells (FIG. 4B).

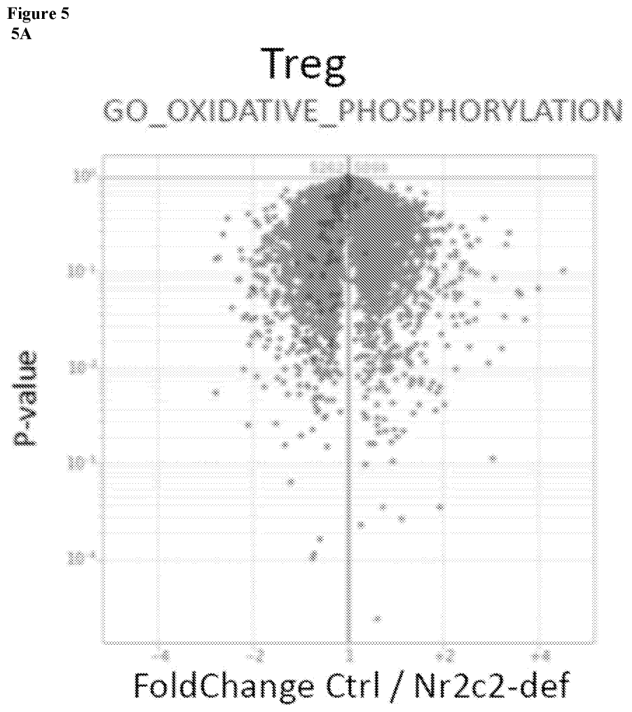

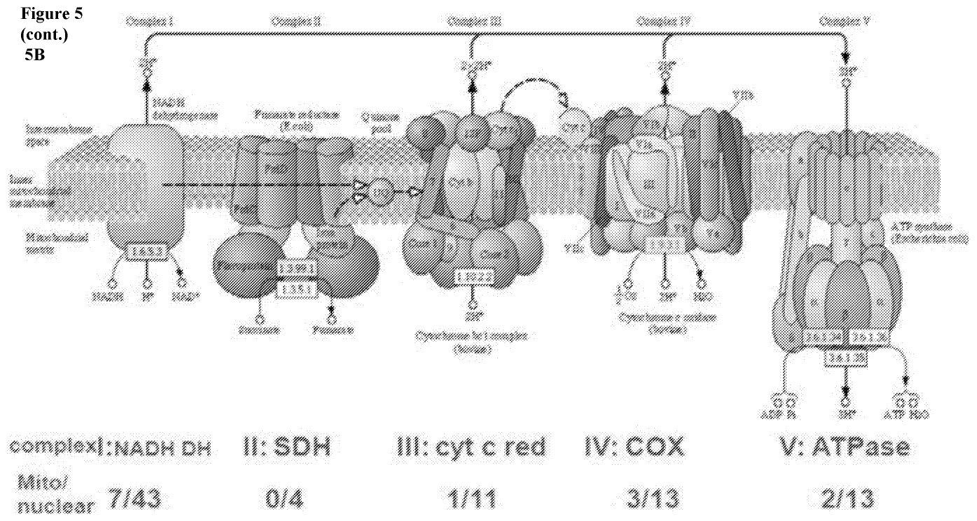

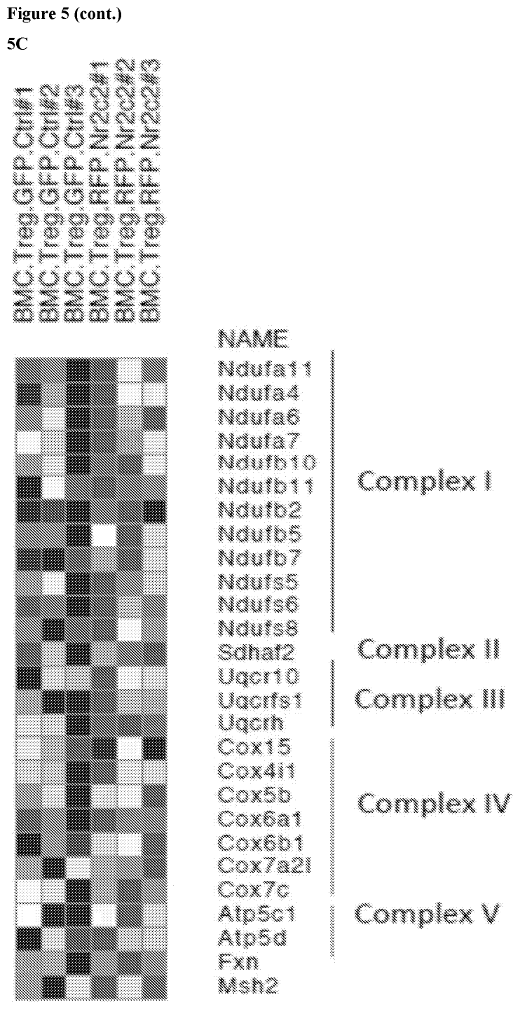

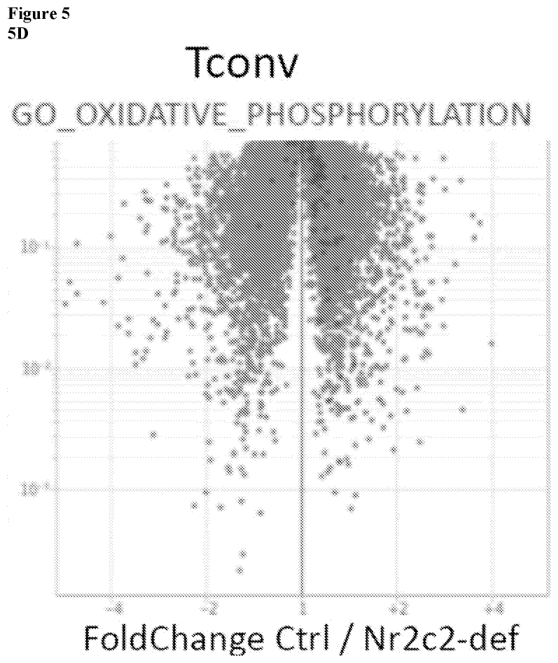

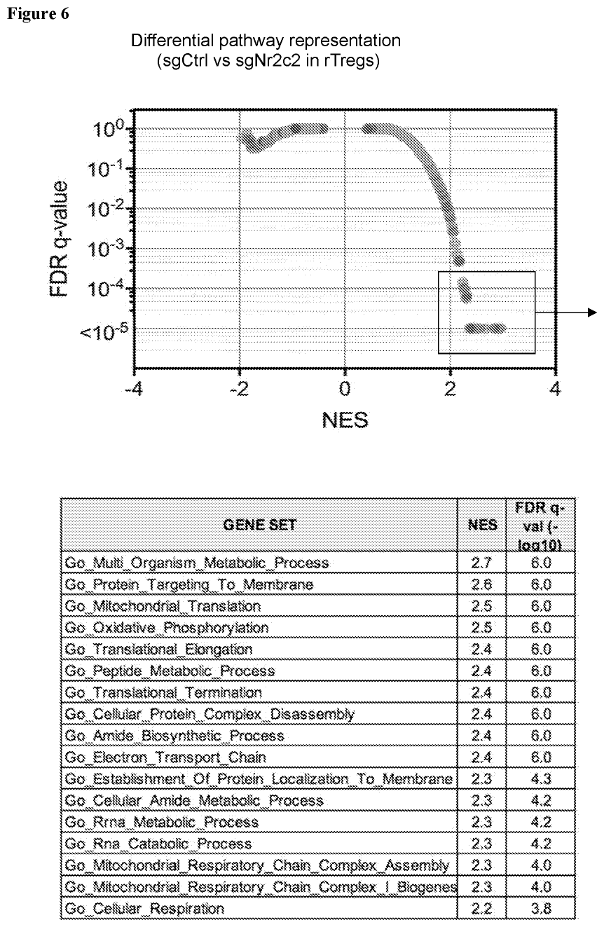

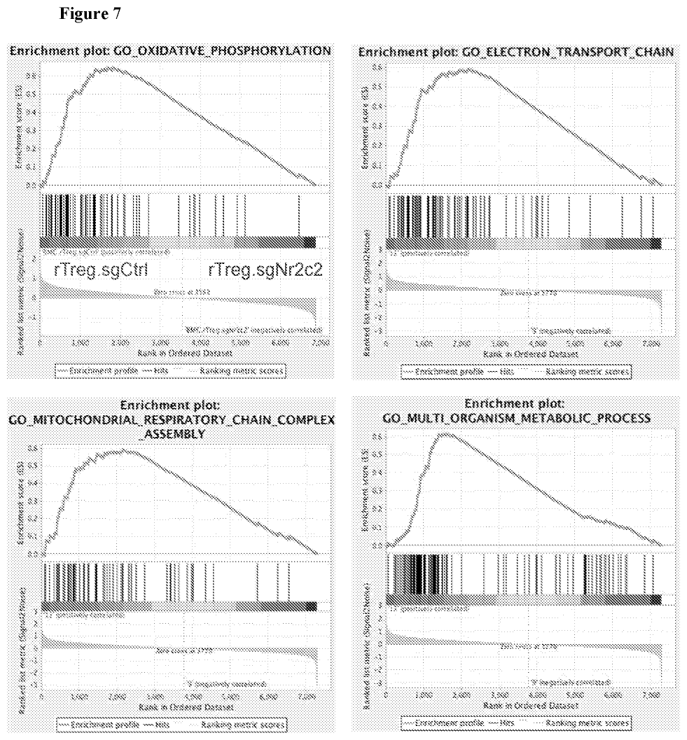

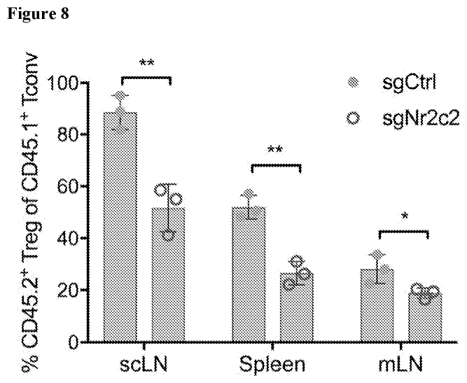

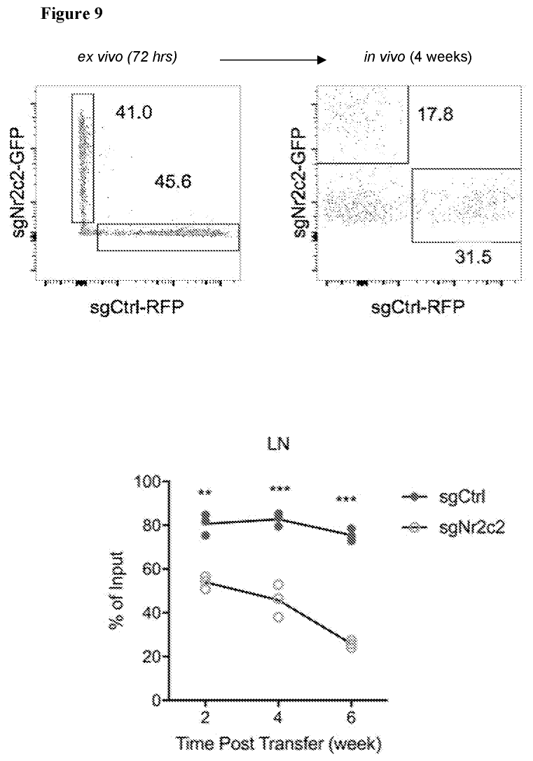

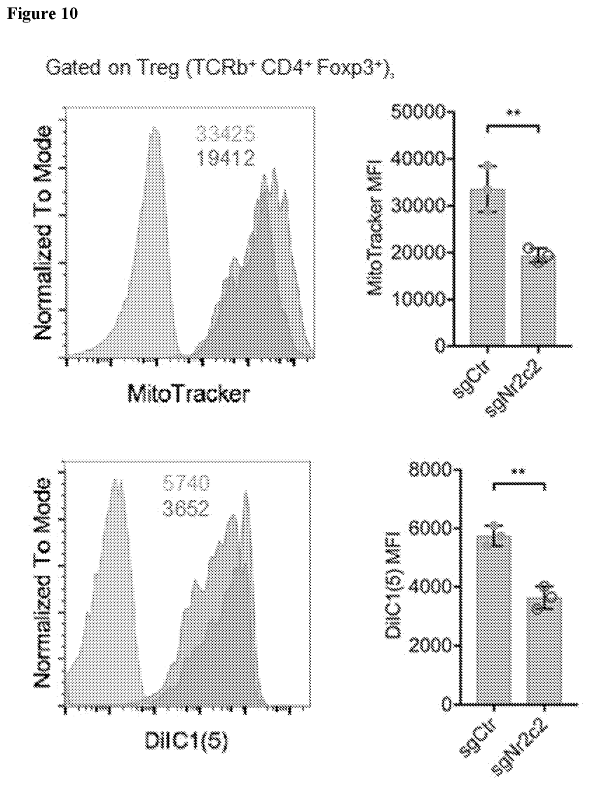

Gene Ontology analysis of these transcriptome data indicated that the transcripts most uniformly altered by the Nr2c2 deficiency in Tregs were those of the mitochondrial oxidative phosphorylation (OXPHOS) respiratory complex (FIG. 5A). Additional genes upregulated or downregulated in Nr2c2-deficient Treg are listed in Tables 3 and 4. Indeed, parsing of the genes that encode the main respiratory chain complexes (schematized for reference in FIG. 5B) showed that transcripts encoding every one of the complexes were induced by the deficiency (FIG. 5C). Importantly, Nr2c2 deficiency had the exact opposite effect in Tconv cells, as the OXPHOS signature was decreased by the editing. These results confirm that even though Nr2c2 is expressed at similar levels in Treg and Tconv cells (per ImmGen database), it has very different functional implications in the two settings.

TABLE 3
Genes upregulated in Nr2c2-deficient Treg

| NAME | Fold Change Ctrl/Nr2c2-deficient Treg |
|---|---|
| 2610307P16Rik | 0.416636591 |
| A530040E14Rik | 0.399253254 |
| Ampd1 | 0.478596842 |
| Bach2os | 0.495474781 |
| Bbs2 | 0.499394768 |
| Capn5 | 0.380669533 |
| Eid1 | 0.469093678 |
| Erbb3 | 0.490352028 |
| Fasl | 0.445535694 |
| Fbxl2 | 0.432295263 |
| Gm12607 | 0.475003195 |
| Gm17354 | 0.479257588 |
| Gm37101 | 0.46196984 |
| Gm37660 | 0.484328314 |
| Gm42967 | 0.431172184 |
| Gm43421 | 0.435601472 |
| Gm43604 | 0.463371838 |
| Gm44093 | 0.477668508 |
| Gm44249 | 0.405751961 |
| Gm45041 | 0.275320727 |
| Gm45679 | 0.377547466 |
| Gpsm2 | 0.404029876 |
| Kcnf2 | 0.489745616 |
| Lactb | 0.422336273 |
| Mx2 | 0.487906255 |
| Ntn1 | 0.436688153 |
| Ntn4 | 0.273788915 |
| Nxt2 | 0.499910633 |
| Pbx2 | 0.494635069 |
| Pcdhgb4 | 0.360821423 |
| Pdzd2 | 0.430024937 |
| Prr33 | 0.454710982 |
| Selenon | 0.304503565 |
| Slc19a2 | 0.486369626 |
| Smim13 | 0.392383002 |
| Tulp3 | 0.44102214 |
| Ube4bos1 | 0.396567376 |

TABLE 4
Genes downregulated in Nr2c2-deficient Treg

| NAME | Fold Change Ctrl/Nr2c2-deficient Treg |
|---|---|
| 1810014B01Rik | 2.165291675 |
| 2210408I21Rik | 2.911610633 |
| 6330403K07Rik | 2.214357162 |
| Adam8 | 4.786077171 |
| Aldh7a1 | 2.355792277 |
| Alms1 | 2.111895213 |
| Ankrd6 | 2.228142089 |
| As3mt | 2.01158643 |
| Asb2 | 2.169408251 |
| Atp6v0d2 | 2.526272461 |
| Axl | 2.514698349 |
| Baiap3 | 3.428545726 |
| Ccr2 | 4.71579204 |
| Ccr5 | 2.440410944 |
| Cdc6 | 2.157753325 |
| Cdkl2 | 2.28621024 |
| Cep170b | 2.954699452 |
| Chd3os | 2.164110497 |
| Clip3 | 2.298526811 |
| Cpd | 2.187341723 |
| Crmp1 | 2.446734028 |
| Cul9 | 2.41155894 |
| Cybb | 2.244587821 |
| Dennd2c | 2.466049718 |
| Dsn1 | 2.85046331 |
| Ebi3 | 2.02990125 |
| Eml6 | 2.85050106 |
| Fan1 | 2.363040964 |
| Fosl2 | 2.141741235 |
| Gcnt1 | 2.965138278 |
| Gm12592 | 2.100809252 |
| Gm37334 | 3.517966065 |
| Gm38200 | 2.036557917 |
| Gm38336 | 3.722713273 |
| Gm42683 | 2.267921883 |
| Gm43336 | 2.141663613 |
| Gm44130 | 2.068224999 |
| Gm7967 | 2.05122141 |
| Gnb5 | 2.577140118 |
| Gramd1c | 2.133579086 |
| Guf1 | 2.027477691 |
| Gzmb | 4.77785639 |
| Hic1 | 2.106973027 |
| Hif3a | 2.898000997 |
| Hip1 | 2.232600039 |
| Ighd | 2.841130527 |
| Il10 | 6.573787942 |
| Itgae | 2.166239907 |
| Kif24 | 2.305779292 |
| Lamc1 | 2.077207827 |
| Lancl2 | 2.245883885 |
| Lilrb4a | 2.713324122 |
| Lrrn3 | 3.927930513 |
| Matn2 | 2.33798331 |
| Megf9 | 2.223035414 |
| Naip5 | 2.205799129 |
| Nav2 | 3.97225029 |
| Nebl | 3.508023873 |
| Nr1d1 | 2.41494121 |
| Nr3c2 | 2.056905664 |
| Parp12 | 2.072534798 |
| Pcyt1b | 2.763764279 |
| Penk | 3.668895372 |
| Plk3 | 2.333154589 |
| Plod2 | 2.201652018 |
| Plpp1 | 2.227391358 |
| Podnl1 | 2.110861985 |
| Pon3 | 2.927016523 |
| Prf1 | 2.181480274 |
| Ptpn5 | 2.914963976 |
| Rassf4 | 3.188529116 |
| Rbks | 3.011195046 |
| Rdh10 | 2.442776384 |
| Rin2 | 2.921338675 |
| Rorc | 5.00512967 |
| RP24-178J22.3 | 2.341281514 |
| RP24-325N9.4 | 2.784129338 |
| Rps6kc1 | 2.717072859 |
| S100a11 | 2.226397533 |
| S100a4 | 2.651229264 |
| Sccpdh | 3.025305609 |
| Scpep1 | 3.055254504 |
| Sdc4 | 2.524354896 |
| Sema4f | 2.53744101 |
| Serpina3f | 3.788869667 |
| Spock2 | 2.204022369 |
| Tas1r3 | 2.580045932 |
| Tfpi | 2.125429509 |
| Tigit | 2.477325004 |
| Tjp3 | 2.028249318 |
| Tlr2 | 2.228533904 |
| Tnfrsf8 | 2.523892118 |
| Tnfsf14 | 3.387192637 |
| Tox2 | 2.584210564 |
| Ttc39c | 2.747583887 |
| Ttn | 2.565414368 |
| Zfp109 | 2.732794741 |
| Zfp273 | 2.24119913 |
| Zik1 | 2.667579704 |

### Incorporation by Reference

All publications, including but not limited to patents and patent applications, cited in this specification, to the extent that they provide exemplary procedural or other details supplementary to those set forth herein, are specifically incorporated herein by reference as if each individual publication were specifically and individually indicated to be incorporated by reference herein as though fully set forth.

### Equivalents

Those skilled in the art will recognize, or be able to ascertain using no more than routine experimentation, many equivalents of the specific embodiments of the invention described herein. Such equivalents are intended to be encompassed by the following claims.

1. A method for recovering from, treating, or preventing cancer in a subject in need thereof comprising administering an effective amount of:
(a) an agent that modulates the level of, activity of, or expression of a nuclear receptor subfamily 2, group C, member 2 (Nr2c2), or fragment thereof, or nucleic acid encoding same;
(b) an Nr2c2 variant, or fragment thereof, or nucleic acid encoding same; or
(c) both (a) and (b);
to the subject to thereby modulate T regulatory cell differentiation, function, activity, or maturation, or combination thereof, in the subject.

2. A method for treating or preventing a disorder associated with inflammation in a subject in need thereof comprising administering to the subject an effective amount of:
(a) an agent that modulates the level of, activity of, or expression of Nr2c2, or fragment thereof, or nucleic acid encoding same;
(b) an Nr2c2 variant, or fragment thereof, or nucleic acid encoding same; or
(c) both (a) and (b);
to the subject to thereby modulate T regulatory cell (Treg) differentiation, function, activity, or maturation, or combinations thereof, in the subject.

3. A method of modulating an inflammatory response in a subject in need thereof comprising administering to the subject an effective amount of:
((a) an agent that modulates the level of, activity of, or expression of Nr2c2, or fragment thereof, or nucleic acid encoding same;
(b) an Nr2c2 variant, or fragment thereof, or nucleic acid encoding same; or
(c) both (a) and (b);
to the subject to thereby modulate T regulatory cell differentiation, function, activity, or maturation, or combinations thereof, in the subject.

4. The method of claim 1, wherein the agent inhibits, decreases, suppresses, reduces, knocks down, or depletes, the level of, actitvity of, or expression of the Nr2c2, or a fragment thereof, or a nucleic acid encoding same.

5. The method of claim 1 or 4, wherein Treg differentiation, function, activity, or maturation, or combinations theref, is blocked.

6. The method of claim 2 or 3, wherein the agent activates, initiates, increases, or stimulates, the level of, actitvity of, or expression of the Nr2c2, or a fragment thereof, or a nucleic acid encoding same.

7. The mehod of any one of claim 2, 3, or 6, wherein Treg differentiation, function, activity, or maturation, or combinations thereof, is enhanced.

8. The method of any one of claims 1, 4, and 5, wherein the agent inhibits, decreases, suppresses, reduces, knock downs, or depletes the level of, actitvity of, or expression of the Nr2c2, or homologs thereof, as set forth in Table 1, Table 2, or combinations thereof.

9. The method of claim 8, wherein the agent is an antagonist of Nr2c2.

10. The method of any one of claim 2, 3, 6, or 7, wherein the agent activates, initiates, increases, or stimulates the level of, actitvity of, or expression of the Nr2c2, or homologs thereof, as set forth in Table 1, Table 2, or combinations thereof.

11. The method of claim 10, wherein the agent is an agonist of Nr2c2.

12. The method of claim 11, wherein the agonist of Nr2c2 is a polyunsaturated fatty acid (PUFA), or metabolite thereof.

13. The method of claim 12, wherein the PUFA is selected from omega-3 fatty acid or omega-6 fatty acid.

14. The method of claim 12, werein the PUFA metabolite is selected from the group consisting of 15-hydroxyeico-satetraonic acid (15-HETE), 13-hydroxy octa-deca dieonic acid (13-HODE), and thiazolidinedione (TZD)-rosiglitazone.

15. The method of claim 11, wherein the agonist of Nr2c2 is a retinoid.

16. The method of claim 15, wherein the retinoid is an all-trans-retinoic acid, retinol (ATRA).

17. The method of claim 11, wherein the agonist of Nr2c2 is a keto mycolic acid from Mycobacterium tuberculosis cell wall lipids.

18. The method of claim 11, wherein the agonist of Nr2c2 is y-linoleic acid.

19. The method of any one of claims 1-3, wherein the Nr2c2 variant, or fragment thereof, or nucleic acid encoding same, comprises at least one substitution, mutations, insertion, deletion, or combination thereof, in Nr2c2 as set forth in Table 1, Table 2, or combinations thereof.

20. The method of claim 19, wherein the Nr2c2 variant, or fragment thereof, or nucleic acid encoding same, comprises at least two, three, four, five, six, seven, eight, nine, ten, or more substitution, mutations, insertion, deletion, or combiantions thereof, in Nr2c2 as set forth in Table 1, Table 2, or combinations thereof.

21. The method of claim 1, wherein the Nr2c2 variant, or fragment thereof, or nucleic acid encoding same, is biologically inactive or functionally defective.

22. The method of claim 21, wherein Treg maturation, differentiation, activity, or function, or combination thereof, is inhibited, decreased, suppressed, reduced, knocked down, or depleted.

23. The method of claim 2 or 3, wherein the Nr2c2 variant, or fragment thereof, or nucleic acid encoding same, is biologically active or functionally active.

24. The method of claim 23, wherein Treg maturation, differentiation, activity, or function, or combination thereof, is activated, initiated, increased, or stimulated.

25. The method of claim 1, wherein the agent knocks down, reduces, eliminates, or decreases Nr2c2 gene levels, expression levels, or both.

26. The method of claim 25, wherein the agent is selected from siNA, Clustered Regularly Interspaced Short Palindromic Repeats-Caspase 9 (CRISPR/Cas9), Transcription activator-like effector nucleases (TALEN), or zinc-finger nuclease (ZFN).

27. The method of claim 2, wherein inflammation is decreased.

28. The method of claim 3, where an inflammatory response is depressed or suppressed.

29. The method of claim 1, wherein the cancer is selected from the group consisting of acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hypopharyngeal cancer, intraocular melanoma, islet cell tumors (endocrine pancreas), Kaposi sarcoma, Langerhans cell histiocytosis, laryngeal cancer, leukemia, lung cancer, non-small cell lung cancer, small cell lung cancer, Hodgkin lymphoma, lymphoma, medulloblastoma, medulloepithelioma, melanoma, mesothelioma, mouth cancer, multiple myeloma, nasopharyngeal cancer, neuroblastoma, non-Hodgkin lymphoma, oral cancer, oropharyngeal cancer, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm, pleuropulmonary blastoma, primary central nervous system lymphoma, prostate cancer, rectal cancer, renal cell (kidney) cancer, rhabdomyosarcoma, salivary gland cancer, sarcoma, Ewing sarcoma family of tumors, sarcoma, Sezary syndrome, skin cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

30. The method of claim 3, wherein the disorder associated with inflammation is selected from the group consisting of: septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacteria and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, and pulmonary inflammation.

31. The method of any one of claims 1-30, wherein the agent or Nr2c2 variant is administered to the subject at a dose of between 0.5-5 grams per day.

32. The method of any of claims 1-30, wherein the agent or the Nr2c2 variantis administered in a pharmaceutically effective amount.

33. The method of claim 31, wherein the pharmaceutically effective amount is provided as a pharmaceutical composition in combination with a pharmaceutically-acceptable excipient, diluent, or carrier.

34. The method of any one of claims 1-33, wherein the a) agent is administered simultaneously as the Nr2c2 variant, or fragment thereof, or nucleic acid encoding same; b) agent is administered in combination with Nr2c2 variant, or fragment thereof, or nucleic acid encoding same; c) agent is administered prior to administering the Nr2c2 variant, or fragment thereof, or nucleic acid encoding same; or d) agent is administered subsequently to administering the Nr2c2 variant, or fragment thereof, or nucleic acid encoding same.

35. The method of any of claims 1-34, wherein the subject is a mammal or non-mammal.

36. The method of claim 35, wherein the subject is a human.
